(19) United States
(12) Patent Application Publication
(10) Pub. No.: US 2024/0036064 A1
(43) Pub. Date: Feb. 1, 2024

(54) METHODS AND COMPOSITIONS FOR DETECTING COGNITIVE DISORDER

(71) Applicant: Sanford Burnham Prebys Medical Discovery Institute, La Jolla, CA (US)

(72) Inventor: Evan Y. SNYDER, La Jolla, CA (US)

(21) Appl. No.: 18/361,439

(22) Filed: Jul. 28, 2023

Related U.S. Application Data
(60) Provisional application No. 63/369,868, filed on Jul. 29, 2022.

(51) Int. Cl. G01N 33/68

(52) U.S. Cl. CPC G01N 33/6896; G01N 2440/14; G01N 2800/302

(57) ABSTRACT

Provided are methods and compositions for detecting cognitive disorders such as Schizophrenia. Demonstrated herein is collapsing response mediator protein-2 (CRMP2) as a biomarker for detecting Schizophrenia in peripheral blood sample.

### CROSS-REFERENCE

This application claims the benefit of U.S. Patent Application No. 63/369,868, filed Jul. 29, 2022, the entirely of which is incorporated herein by reference.

### BACKGROUND

Cognitive disorders are neurological disorders. Patients with cognitive disorders are no longer fully oriented to time and space. Cognitive disorder can be caused by malfunctions or abnormalities of the brain, peripheral and central nervous system. These malfunctions can be related to structural, biochemical, or electrical abnormalities, which in some cases can affect behavior, body control, and memory of the patients. The burden of deaths and disabilities caused by cognitive disorders is recognized as a global public health challenge. Thus, early diagnostic aid to identify patients with cognitive disorders such as schizophrenia (SCZ) is needed.

### SUMMARY OF THE INVENTION

Collapsin response mediator protein 2 (CRMP2) is a master regulator of axon guidance, dendritic branching, and spine formation: hence, a neural network modulator. The abundance of active CRMP2 and insufficiency of opposing inactive phosphorylated CRMP2 (p-CRMP2) likely disrupts neuronal function. In one aspect, the present disclosure provides a method for determining an increased risk for a cognitive disorder in a human subject, comprising: collecting a biological sample from the human subject; determining, by an assay, a level of collapsing response mediator protein-2 (CRMP2) in the biological sample, wherein the assay comprises: i. contacting the biological sample with an agent that recognizes CRMP2, and ii. measuring the level of bound CRMP2 and thereby determining the level of CRMP2 present in the biological sample; and detecting the level of CRMP2. In some embodiments, the assay further comprises: i. contacting the biological sample with an agent that recognizes phosphorylated CRMP2 (p-CRMP2), measuring a level of bound p-CRMP2 and thereby determining the level of p-CRMP2 present in the biological sample; and iii. detecting the level of p-CRMP2. In some embodiments, the method further comprises: a. computing a ratio of the p-CRMP2 to CRMP2 (p-CRMP2:CRMP2 ratio); and b. detecting a reduced p-CRMP2:CRMP2 ratio in comparison to a reference p-CRMP2:CRMP2 ratio.

In some embodiments, the cognitive disorder is not age-related. In some embodiments, the cognitive disorder is Schizophrenia (SCZ). In some embodiments, the SCZ is an early stage of SCZ.

In some embodiments, the biological sample is a blood, saliva, urine, serum, tears, skin, tissue, or hair from the human subject. In some embodiments, the biological sample is a blood sample from the human subject comprising peripheral blood mononuclear cells (PBMC). In some embodiments, the PBMC comprises lymphocytes.

In some embodiments, the human subject is a male human. In some embodiments, the human subject is less than 40 years old. In some embodiments, the human subject is less than 30 years old. In some embodiments, the human subject is greater than 17 years old. In some embodiments, the human subject is greater than 18 years old.

In some embodiments, the human subject experiences one or more symptoms of the cognitive disorder comprises delusion, hallucination, disorganized thinking, disorganized speech, extremely disorganized or abnormal motor behavior, or social withdrawal.

In some embodiments, the assay is a proteomic assay. In some embodiments, the proteomic assay is immunoassay, mass spectrometry, or intracellular flow cytometry. In some embodiments, the immunoassay is selected from western blotting, dot blotting, quantitative enzyme-linked immunosorbent assays (ELISA), immunocytochemistry (ICC), immunohistochemistry (IHC), protein multiplex assay, or lateral flow test.

In some embodiments, the agent is an antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody.

In some embodiments, the method further comprises identifying the human subject with low added to high added risk of having the cognitive disorder if the p-CRMP2:CRMP2 ratio of the subject is decreased compared to the reference p-CRMP2:CRMP2 ratio. In some embodiments, the method further comprises identifying the human subject with added risk of having the cognitive disorder if the p-CRMP2:CRMP2 ratio of the subject is lower than 1.0, 0.9, 0.7, 0.6, 0.5, 0.4, 0.3, 0.2, 0.1 or within a range defined by any of the preceding values. In some embodiments, the method further comprises identifying the human subject with added risk of having the cognitive disorder if the p-CRMP2:CRMP2 ratio of the subject is between 0.1 to 0.4. In some embodiments, the method further comprises identifying the human subject with added risk of having the cognitive disorder if the p-CRMP2:CRMP2 ratio of the subject is between 0.2 to 0.5. In some embodiments, the method further comprises identifying the human subject with added risk of having the cognitive disorder if the p-CRMP2:CRMP2 ratio of the subject is about 40% lower than the reference p-CRMP2:CRMP2 ratio.

In some embodiments, the CRMP2 is phosphorylated at Serine 522 (p-S522-CRMP2). In some embodiments, measuring the level of p-CRMP2 comprises measuring a phosphorylation of CRMP2 at Serine 522.

In some embodiments, the human subject with added risk of having the cognitive disorder is further identified with one or more severe symptoms of the cognitive disorder. In some embodiments, the human subject with added risk of having the cognitive disorder is further identified with one or more moderate symptoms of the cognitive disorder. In some embodiments, the human subject with added risk of having the cognitive disorder is further identified with one or more mild symptoms of the cognitive disorder.

In some embodiments, the assay comprises a protein panel, wherein CRMP2 and p-CRMP2 are part of the protein panel. In some embodiments, the subject is tested for at least 2 other proteins in addition to CRMP2 and p-CRMP2.

In some embodiments, the method further comprises sequencing a genome of the human subject. In some embodiments, the method further comprises testing DNA, mRNA, or cDNA of the subject to detect other biomarkers.

In some embodiments, the method further comprises monitoring the levels of CRMP2 and p-CRMP2 from the human subject over a period of at least 1 year. In some embodiments, the method further comprises retesting the levels of CRMP2 and p-CRMP2 from the subject if the p-CRMP2:CRMP2 ratio of the human subject is between 0.1 and 1.0.

In some embodiments, the method further comprises treating the human subject with one or more antipsychotic drugs. In some embodiments, the method further comprises monitoring the human subject for SCZ symptoms. In some embodiments, the method further comprises treating positive symptoms of SCZ in the human subject. In some embodiments, the method further comprises treating negative symptoms of SCZ in the human subject. In some embodiments, the method further comprises treating cognitive impairment of SCZ in the human subject. In some embodiments, the method further comprises treating one or more symptoms of SCZ in the human subject using a D-amino acid oxidase (DAAO) inhibitor. In some embodiments, the method further comprises treating one or more symptoms of SCZ in the human subject using a vesicular monoamine transporter 2 (VMAT2) inhibitor. In some embodiments, the method further comprises treating one or more symptoms of SCZ in the human subject using a muscarinic M4 agonist.

In another aspect, the present disclosure provides a method comprising: collecting a biological sample from a human subject; determining, by an assay, a level of CRMP2 in the biological sample from the subject, wherein the assay comprises: i. contacting the biological sample with an agent that recognizes CRMP2, ii. measuring the level of bound CRMP2 and thereby determining the level of CRMP2 present in the biological sample, and iii. computing the risk of the human subject having a cognitive disorder based on determining the level of CRMP2 in comparison to a reference level of CRMP2; and administering a therapeutic agent to the human subject, wherein the therapeutic agent is configured to mitigate or alleviate one or more symptoms of the cognitive disorder in the human subject. In some embodiments, the therapeutic agent is configured to modulate the level of CRMP2.

In another aspect, the present disclosure provides a method of treating a cognitive disorder in a human subject, comprising: performing the method as disclosed and described herein; and administering a therapeutic agent or clinical investigational product to the human subject. In some embodiments, the therapeutic agent or clinical investigational product is pimavanserin, haloperidol, loxapine, thioridazine, molindone, thiothixene, fluphenazine, mesoridazine, trifluoperazine, perphenazine, chlorpromazine, flupentixol, levomepromazine, periciazine, pimozide, prochlorperazine, promazine, sulpiride, zuchlopenthixol. aripiprazole, clozapine, ziprasidone, risperidone, quetiapine, olanzapine, asenapine, iloperidone, amisulpride, lurasidone, or paliperidone. In some embodiments, the therapeutic agent or clinical investigational product is pimavanserin. In some embodiments, the therapeutic agent or clinical investigational product is a D-amino acid oxidase (DAAO) inhibitor, vesicular monoamine transporter 2 (VMAT2) inhibitor, or muscarinic M4 agonist. In some embodiments, the DAAO inhibitor is Luvadaxistat, the VMAT2 inhibitor is Valbenazine, Tetrabenazine or Deutetrabenazine, and the muscarinic M4 agonist is NBI-1117568. In some embodiments, the human subject is selected for treatment if the p-CRMP2:CRMP2 ratio of the subject is lower than 1.0, 0.9, 0.8, 0.7, 0.6, 0.5, 0.4, 0.3, 0.2, 0.1 or within a range defined by any of the preceding values. In some embodiments, the cognitive disorder is not age-related. In some embodiments, the cognitive disorder is SCZ. In some embodiments, the SCZ is an early stage of SCZ. In some embodiments, the SCZ is treatment resistant SCZ.

In some embodiments, the biological sample is a blood, saliva, urine, serum, tears, skin, tissue, or hair from the subject. In some embodiments, the biological sample is a blood sample from the subject comprising peripheral blood mononuclear cells (PBMC). In some embodiments, the PBMC comprises lymphocytes.

In some embodiments, the human subject in need thereof is less than 40 years old. In some embodiments, the human subject in need thereof is less than 30 years old. In some embodiments, the human subject in need thereof is greater than 17 years old. In some embodiments, wherein the human subject in need thereof is greater than 18 years old.

In some embodiments, the assay further comprises: a. contacting the biological sample with an agent that recognizes phosphorylated CRMP2 (p-CRMP2), and b. measuring a level of bound p-CRMP2 and thereby determining the level of p-CRMP2 present in the biological sample. In some embodiments, the method further comprises: a. computing a ratio of the p-CRMP2 to CRMP2 (p-CRMP2:CRMP2 ratio); and b. detecting a reduced p-CRMP2:CRMP2 ratio in comparison to a reference p-CRMP2:CRMP2 ratio.

In some embodiments, the assay is a proteomic assay. In some embodiments, the proteomic assay is immunoassay, mass spectrometry, or intracellular flow cytometry. In some embodiments, the immunoassay is selected from western blotting, dot blotting, quantitative enzyme-linked immunosorbent assays (ELISA), immunocytochemistry (ICC), immunohistochemistry (IHC), protein multiplex assay, or lateral flow test.

In some embodiments, the agent is an antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody.

In some embodiments, the assay comprises a protein panel, wherein the CRMP2 and p-CRMP2 are part of the protein panel. In some embodiments, measuring the level of p-CRMP2 comprises measuring a phosphorylation of CRMP2 at Serine 522.

In some embodiments, one or more symptoms of the cognitive disorder comprises delusion, hallucination, disorganized thinking, disorganized speech, extremely disorganized or abnormal motor behavior, or social withdrawal.

In some embodiments, the method further comprises monitoring the levels of CRMP2 and p-CRMP2 from the human subject in need thereof over a period of at least 1 year.

In some embodiments, the method further comprises administering Clinical Assessment Interview for Negative Symptoms (CAINS), Brief Negative Symptom Scale (BNSS), Positive and Negative Symptoms Scale (PANSS), Scale for the Assessment of Positive Symptoms (SAPS), Scale for the Assessment of Negative Symptoms (SANS), Negative Symptom Assessment-16 (NSA-16), Clinical Global Impression Schizophrenia (CGI-SCH) or combination of two or more of the preceding to the human subject. In some embodiments, the method further comprises administering a PANSS to the human subject.

In some embodiments, the method further comprises administering a therapeutic agent or clinical investigational product to the human subject. In some embodiments, the therapeutic agent or clinical investigational product is pimavanserin, haloperidol, loxapine, thioridazine, molindone, thiothixene, fluphenazine, mesoridazine, trifluoperazine, perphenazine, chlorpromazine, flupentixol, levomepromazine, periciazine, pimozide, prochlorperazine, promazine, sulpiride, zuchlopenthixol. aripiprazole, clozapine, ziprasidone, risperidone, quetiapine, olanzapine, asenapine, iloperidone, amisulpride, lurasidone, or paliperidone. In some embodiments, the therapeutic agent or clinical investigational product is pimavanserin. In some embodiments, the therapeutic agent or clinical investigational product is a D-amino acid oxidase (DAAO) inhibitor, vesicular monoamine transporter 2 (VMAT2) inhibitor, or muscarinic M4 agonist. In some embodiments, the DAAO inhibitor is Luvadaxistat, the VMAT2 inhibitor is Valbenazine, Tetrabenazine or Deutetrabenazine, and the muscarinic M4 agonist is NBI-1117568.

In some embodiments, the cognitive disorder is treatment resistant SCZ.

In some embodiments, the present disclosure provides a method of treating a cognitive disorder in a human subject comprising: performing the method as described herein and administering a therapeutic agent or clinical investigational product to the human subject. In some embodiments, the therapeutic agent or clinical investigational product is pimavanserin, haloperidol, loxapine, thioridazine, molindone, thiothixene, fluphenazine, mesoridazine, trifluoperazine, perphenazine, chlorpromazine, flupentixol, levomepromazine, periciazine, pimozide, prochlorperazine, promazine, sulpiride, zuchlopenthixol. aripiprazole, clozapine, ziprasidone, risperidone, quetiapine, olanzapine, asenapine, iloperidone, amisulpride, lurasidone, or paliperidone. In some embodiments, the therapeutic agent or clinical investigational product is pimavanserin. In some embodiments, the therapeutic agent or clinical investigational product is a D-amino acid oxidase (DAAO) inhibitor, vesicular monoamine transporter 2 (VMAT2) inhibitor, or muscarinic M4 agonist. In some embodiments, the DAAO inhibitor is Luvadaxistat, the VMAT2 inhibitor is Valbenazine, Tetrabenazine or Deutetrabenazine, and the muscarinic M4 agonist is NBI-1117568. In some embodiments, the human subject is selected for treatment if the p-CRMP2:CRMP2 ratio of the subject is lower than 1.0, 0.9, 0.8, 0.7, 0.6, 0.5, 0.4, 0.3, 0.2, 0.1 or within a range defined by any of the preceding values. In some embodiments, the cognitive disorder is treatment resistant SCZ.

In one aspect, the present disclosure provides a kit comprising: a. one or more agents to collect a biological sample from a human subject; b. one or more agents to measure a level of CRMP2 and a level of p-CRMP2 from the biological sample obtained from the subject; and c. an instruction to collect the biological sample and measure the levels of CRMP2 and p-CRMP2 using the agents in the kit.

In some embodiments, the agents to measure the levels of CRMP2 and p-CRMP2 are antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody. In some embodiments, the anti-p-CRMP2 is an anti-p-Serine 522-CRMP2 (p-S522-CRMP2).

In some embodiments, the biological sample is a blood, saliva, urine, serum, tears, skin, tissue, biopsy, or hair from the subject. In some embodiments, the blood sample comprises peripheral blood mononuclear cells (PBMC). In some embodiments, the PBMC comprises lymphocytes.

In some embodiments, the subject less than 40 years old. In some embodiments, the subject is less than 30 years old. In some embodiments, the subject is greater than 17 years. In some embodiments, the subject is greater than 18 years old.

In some embodiments, the kit is a lateral flow test. In some embodiments, the kit comprises a protein panel, wherein CRMP2 and p-CRMP2 are part of the protein panel.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B show representative brightfield photomicrographs of the basilar dendrites of Golgi-stained pyramidal neurons in the DLPFC from a representative control subject (FIG. 1A) compared with a representative patient with SCZ (FIG. 1B). Red arrows point to dendritic spines, which are markedly reduced in the SCZ patient.

FIGS. 2A-2B show authentication of the sensitivity and specificity of the antibodies against collapsin response mediator protein 2 (CRMP2; FIG. 2A) and the phosphorylated form of CRMP2 (FIG. 2B) in brain lysate and PBMC samples based on immunoblot analysis.

FIGS. 3A-3B show validation of anti-CRMP1 antibodies using immunoblotting. FIG. 3A shows immunoblot analysis using 2 different anti-CRMP1 antibodies (2E7G and 2C6G) against wild (WT) and crmp1−/− (CRMP1 KO) or crmp2−/− (CRMP2 KO) mouse brain lysates. FIG. 3B shows immunoblot analysis of human peripheral blood mononuclear cell (PBMC) fraction (specifically, lymphocytes). The differences seen in SCZ vs. unaffected patients pivoted on differences in the abundance of the CRMP2 isoform rather than of the CRMP1 isoform was proven through the use of antibodies against CRMP1.

FIG. 4 shows that the level of CRMP2 in brains of patients with SCZ compared with those of unaffected age-matched controls is elevated.

FIGS. 5A-5B show although CRMP2 levels were significantly higher in brains from SCZ patients compared to those from unaffected controls as seen in FIG. 4, the p-CRMP2 protein expression in those SCZ brains in FIG. 5A and, hence, the ratio of p-CRMP2:CRMP2 protein expression in FIG. 5B initially appeared not to be significantly different from controls (p>0.05) when the cohorts were examined in the aggregate.

FIGS. 6A-6B show the p-CRMP2:CRMP2 ratio in lymphocytes from patients with SCZ was significantly lower than in lymphocytes from unaffected age-matched control human subjects.

### DETAILED DESCRIPTION OF THE INVENTION

Provided herein are methods and kits for early detection of cognitive disorders such as Schizophrenia (SCZ). In one aspect, the present disclosure provides a method for determining an increased risk for a cognitive disorder in a human subject, comprising: collecting a biological sample from the human subject; determining, by an assay, a level of collapsing response mediator protein-2 (CRMP2) in the biological sample, wherein the assay comprises: i. contacting the biological sample with an agent that recognizes CRMP2, and ii. measuring the level of bound CRMP2 and thereby determining the level of CRMP2 present in the biological sample; and detecting the level of CRMP2. In another aspect, the present disclosure provides a method for determining an increased risk for a cognitive disorder in a human subject, comprising: collecting a biological sample from the human subject; determining, by an assay, a level of collapsing response mediator protein-2 (CRMP2) in the biological sample, wherein the assay comprises: i. contacting the biological sample with an agent that recognizes CRMP2, and ii. measuring the level of bound CRMP2 and thereby determining the level of CRMP2 present in the biological sample; and comparing the level of CRMP2 to a reference level of CRMP2, wherein the reference level of CRMP2 is derived from a group of subjects known not to have the cognitive disorder, and wherein the human subject is identified with the increased risk for the cognitive disorder if the level of CRMP2 is increased when compared to the reference level of CRMP2.

In some embodiments, the assay further comprises: i. contacting the biological sample with an agent that recognizes phosphorylated CRMP2 (p-CRMP2), ii. measuring a level of bound p-CRMP2 and thereby determining the level of p-CRMP2 present in the biological sample; and iii. detecting the level of p-CRMP2. In some embodiments, the method further comprises: a. computing a ratio of the p-CRMP2 to CRMP2 (p-CRMP2:CRMP2 ratio); and b. detecting a reduced p-CRMP2:CRMP2 ratio in comparison to a reference p-CRMP2:CRMP2 ratio. In various embodiments, the method further comprises a. computing a ratio of the p-CRMP2 to CRMP2 (p-CRMP2:CRMP2 ratio); and b. comparing the p-CRMP2:CRMP2 ratio to a reference p-CRMP2:CRMP2 ratio; wherein the reference p-CRMP2:CRMP2 ratio is derived from a second group of subjects known not to have the cognitive disorder.

In some embodiments, the cognitive disorder is not age-related. In some embodiments, the cognitive disorder is SCZ. In some embodiments, the SCZ is an early stage of SCZ. In some embodiments, the SCZ is treatment resistant SCZ.

In some embodiments, the biological sample is a blood, saliva, urine, serum, tears, skin, tissue, or hair from the human subject. In some embodiments, the biological sample is a blood sample from the human subject comprising peripheral blood mononuclear cells (PBMC). In some embodiments, the PBMC comprises lymphocytes.

In some embodiments, the human subject is a male. In some embodiments, the human subject is a female. In some embodiments, the human subject is less than 40 years old. In some embodiments, the human subject is less than 30 years old. In some embodiments, the human subject is greater than 17 years old. In some embodiments, the human subject is greater than 18 years old.

In some embodiments, the human subject experiences one or more symptoms of the cognitive disorder comprises delusion, hallucination, disorganized thinking, disorganized speech, extremely disorganized or abnormal motor behavior, or social withdrawal.

In some embodiments, the assay is a proteomic assay. In some embodiments, the proteomic assay is immunoassay, mass spectrometry, or intracellular flow cytometry. In some embodiments, the immunoassay is selected from western blotting, dot blotting, quantitative enzyme-linked immunosorbent assays (ELISA), immunocytochemistry (ICC), immunohistochemistry (IHC), protein multiplex assay, or lateral flow test.

In some embodiments, the agent is an antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody.

In some embodiments, the method further comprises identifying (e.g., diagnosing) the human subject with high added risk of having the cognitive disorder if the p-CRMP2:CRMP2 ratio of the biological sample in the subject is decreased compared to the reference p-CRMP2:CRMP2 ratio (e.g., of a non-diseased subject). In some embodiments, the method further comprises identifying the human subject with added risk of having the cognitive disorder if the p-CRMP2:CRMP2 ratio of the subject is lower than at least or up to about 1.0, at least or up to about 0.9, at least or up to about 0.8, at least or up to about 0.7, at least or up to about 0.6, at least or up to about 0.5, at least or up to about 0.4, at least or up to about 0.3, at least or up to about 0.2, at least or up to about 0.1 or within a range defined by any of the preceding values. In some embodiments, the method further comprises identifying (e.g., diagnosing) the human subject with added risk of having the cognitive disorder when the p-CRMP2:CRMP2 ratio of the biological sample in the subject is decreased by at least or up to about 0.1-fold, at least or up to about 0.2-fold, at least or up to about 0.3-fold, at least or up to about 0.4-fold, at least or up to about 0.5-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 12-fold, at least or up to about 15-fold, at least or up to about 20-fold, at least or up to about 25-fold, at least or up to about 30-fold, at least or up to about 35-fold, at least or up to about 40-fold, at least or up to about 45-fold, at least or up to about 50-fold, or at least or up to about 100-fold compared to that of a predetermined reference value (or that of a non-diseased subject).

In some embodiments, the method further comprises identifying (e.g., diagnosing) the human subject with added risk of having the cognitive disorder if the p-CRMP2:CRMP2 ratio of the biological sample in the subject is between about 0.1 to about 0.4. In some embodiments, the method further comprises identifying (e.g., diagnosing) the human subject with added risk of having the cognitive disorder if the p-CRMP2:CRMP2 ratio of the biological sample in the subject is between about 0.2 to about 0.5. In some embodiments, the method further comprises identifying (e.g., diagnosing) the human subject with added risk of having the cognitive disorder if the p-CRMP2:CRMP2 ratio of the biological sample in the subject is at least or up to about 60%, at least or up to about 50% at least or up to about 40%, at least or up to about 30%, at least or up to about 20%, at least or up to about 10%, at least or up to about 5%, or at least or up to about 3% lower than the reference p-CRMP2:CRMP2 ratio (e.g., of a non-diseased subject).

In some embodiments, the method further comprises identifying (e.g., diagnosing) the human subject with added risk of having the cognitive disorder when the CRMP2 measurement of the biological sample in the subject is increased by at least or up to about 0.1-fold, at least or up to about 0.2-fold, at least or up to about 0.3-fold, at least or up to about 0.4-fold, at least or up to about 0.5-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 12-fold, at least or up to about 15-fold, at least or up to about 20-fold, at least or up to about 25-fold, at least or up to about at least or up to about 35-fold, at least or up to about 40-fold, at least or up to about 45-fold, at least or up to about 50-fold, or at least or up to about 100-fold compared to that of a predetermined reference value (or that of a non-diseased subject).

In some embodiments, the CRMP2 is phosphorylated at Serine 522 (p-S522-CRMP2). In some embodiments, measuring the level of p-CRMP2 comprises measuring a phosphorylation of CRMP2 at Serine 522.

In some embodiments, the human subject with added risk of having the cognitive disorder is further identified with one or more severe symptoms of the cognitive disorder. In some embodiments, the human subject with added risk of having the cognitive disorder is further identified with one or more moderate symptoms of the cognitive disorder. In some embodiments, the human subject with added risk of having the cognitive disorder is further identified with one or more mild symptoms of the cognitive disorder.

In some embodiments, the assay comprises a protein panel, wherein CRMP2 and p-CRMP2 are part of the protein panel. In some embodiments, the subject is tested for at least 2 other proteins in addition to CRMP2 and p-CRMP2.

In some embodiments, the method further comprises sequencing a genome of the human subject. In some embodiments, the method further comprises testing DNA, mRNA, or cDNA of the subject to detect other biomarkers.

In some embodiments, the method further comprises monitoring the levels of CRMP2 and p-CRMP2 from the human subject over a period of at least 1 year. In some embodiments, the method further comprises retesting the levels of CRMP2 and p-CRMP2 from the subject if the p-CRMP2:CRMP2 ratio of the human subject is between about 0.1 to about 1.0. In some embodiments, the method further comprises retesting the levels of CRMP2 and p-CRMP2 from the subject if the p-CRMP2:CRMP2 ratio of the human subject is between about 0.4 to about 1.0.

In some embodiments, the method further comprises treating the human subject with one or more antipsychotic drugs. In some embodiments, the method further comprises monitoring the human subject for SCZ symptoms. In some embodiments, the method further comprises treating positive symptoms of SCZ in the human subject. In some embodiments, the method further comprises treating negative symptoms of SCZ in the human subject. In some embodiments, the method further comprises treating cognitive impairment of SCZ in the human subject. In some embodiments, the method further comprises treating one or more symptoms of SCZ in the human subject using a D-amino acid oxidase (DAAO) inhibitor. In some embodiments, the method further comprises treating one or more symptoms of SCZ in the human subject using a vesicular monoamine transporter 2 (VMAT2) inhibitor. In some embodiments, the method further comprises treating one or more symptoms of SCZ in the human subject using a muscarinic M4 agonist.

In another aspect, the present disclosure provides a method comprising: collecting a biological sample from a human subject; determining, by an assay, a level of CRMP2 in the biological sample from the subject, wherein the assay comprises: i. contacting the biological sample with an agent that recognizes CRMP2, ii. measuring the level of bound CRMP2 and thereby determining the level of CRMP2 present in the biological sample, and iii. computing the risk of the human subject having a cognitive disorder based on determining the level of CRMP2 in comparison to a reference level of CRMP2; and administering a therapeutic agent to the human subject, wherein the therapeutic agent is configured to mitigate or alleviate one or more symptoms of the cognitive disorder in the human subject. In some embodiments, the therapeutic agent is configured to modulate the level of CRMP2.

In some embodiments, the cognitive disorder is not age-related. In some embodiments, the cognitive disorder is SCZ. In some embodiments, the SCZ is an early stage of SCZ. In some embodiments, the SCZ is treatment resistant SCZ.

In some embodiments, the biological sample is a blood, saliva, urine, serum, tears, skin, tissue, or hair from the subject. In some embodiments, the biological sample is a blood, saliva, or urine from the subject. In some embodiments, the biological sample is a blood sample from the subject comprising peripheral blood mononuclear cells (PBMC). In some embodiments, the PBMC comprises lymphocytes.

In some embodiments, the human subject in need thereof is less than 40 years old. In some embodiments, the human subject in need thereof is less than 30 years old. In some embodiments, the human subject in need thereof is greater than 17 years old. In some embodiments, wherein the human subject in need thereof is greater than 18 years old.

In some embodiments, the assay further comprises: a. contacting the biological sample with an agent that recognizes phosphorylated CRMP2 (p-CRMP2), and b. measuring a level of bound p-CRMP2 and thereby determining the level of p-CRMP2 present in the biological sample. In some embodiments, the method further comprises: a. computing a ratio of the p-CRMP2 to CRMP2 (p-CRMP2:CRMP2 ratio); and b. detecting a reduced p-CRMP2:CRMP2 ratio in comparison to a reference p-CRMP2:CRMP2 ratio. In some embodiments, the method further comprises: computing a ratio of the p-CRMP2 to CRMP2 (p-CRMP2:CRMP2 ratio); and comparing the p-CRMP2:CRMP2 ratio to a reference p-CRMP2:CRMP2 ratio; wherein the reference p-CRMP2:CRMP2 ratio is derived from a second group of subjects known not to have the cognitive disorder.

In some embodiments, the assay is a proteomic assay. In some embodiments, the proteomic assay is immunoassay, mass spectrometry, or intracellular flow cytometry. In some embodiments, the immunoassay is selected from western blotting, dot blotting, quantitative enzyme-linked immunosorbent assays (ELISA), immunocytochemistry (ICC), immunohistochemistry (IHC), protein multiplex assay, or lateral flow test.

In some embodiments, the agent is an antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody.

In some embodiments, the assay comprises a protein panel, wherein the CRMP2 and p-CRMP2 are part of the protein panel. In some embodiments, measuring the level of p-CRMP2 comprises measuring a phosphorylation of CRMP2 at Serine 522.

In some embodiments, one or more symptoms of the cognitive disorder comprises delusion, hallucination, disorganized thinking, disorganized speech, extremely disorganized or abnormal motor behavior, or social withdrawal.

In some embodiments, the method further comprises monitoring the levels of CRMP2 and p-CRMP2 from the human subject in need thereof over a period of at least 1 year.

In some embodiments, the method further comprises administering Clinical Assessment Interview for Negative Symptoms (CAINS), Brief Negative Symptom Scale (BNSS), Positive and Negative Symptoms Scale (PANSS), Scale for the Assessment of Positive Symptoms (SAPS), Scale for the Assessment of Negative Symptoms (SANS), Negative Symptom Assessment-16 (NSA-16), Clinical Global Impression Schizophrenia (CGI-SCH) or combination of two or more of the preceding to the human subject. In some embodiments, the method further comprises administering a PANSS to the human subject.

In some embodiments, the method further comprises administering a therapeutic agent or clinical investigational product to the human subject. In some embodiments, the therapeutic agent or clinical investigational product is pimavanserin, haloperidol, loxapine, thioridazine, molindone, thiothixene, fluphenazine, mesoridazine, trifluoperazine, perphenazine, chlorpromazine, flupentixol, levomepromazine, periciazine, pimozide, prochlorperazine, promazine, sulpiride, zuchlopenthixol. aripiprazole, clozapine, ziprasidone, risperidone, quetiapine, olanzapine, asenapine, iloperidone, amisulpride, lurasidone, or paliperidone. In some embodiments, the therapeutic agent or clinical investigational product is pimavanserin. In some embodiments, the therapeutic agent or clinical investigational product is a D-amino acid oxidase (DAAO) inhibitor, vesicular monoamine transporter 2 (VMAT2) inhibitor, or muscarinic M4 agonist. In some embodiments, the DAAO inhibitor is Luvadaxistat, the VMAT2 inhibitor is Valbenazine, Tetrabenazine or Deutetrabenazine, and the muscarinic M4 agonist is NBI-1117568.

In some embodiments, the cognitive disorder is treatment resistant SCZ.

In some embodiments, the present disclosure provides a method of treating a cognitive disorder in a human subject comprising: performing the method as described herein and administering a therapeutic agent or clinical investigational product to the human subject. In some embodiments, the therapeutic agent or clinical investigational product is pimavanserin, haloperidol, loxapine, thioridazine, molindone, thiothixene, fluphenazine, mesoridazine, trifluoperazine, perphenazine, chlorpromazine, flupentixol, levomepromazine, periciazine, pimozide, prochlorperazine, promazine, sulpiride, zuchlopenthixol. aripiprazole, clozapine, ziprasidone, risperidone, quetiapine, olanzapine, asenapine, iloperidone, amisulpride, lurasidone, or paliperidone. In some embodiments, the therapeutic agent or clinical investigational product is pimavanserin. In some embodiments, the therapeutic agent or clinical investigational product is a D-amino acid oxidase (DAAO) inhibitor, vesicular monoamine transporter 2 (VMAT2) inhibitor, or muscarinic M4 agonist. In some embodiments, the DAAO inhibitor is Luvadaxistat, the VMAT2 inhibitor is Valbenazine, Tetrabenazine or Deutetrabenazine, and the muscarinic M4 agonist is NBI-1117568. In some embodiments, the human subject is selected for treatment if the p-CRMP2:CRMP2 ratio of the subject is lower than 1.0, 0.9, 0.8, 0.7, 0.6, 0.5, 0.4, 0.3, 0.2, 0.1 or within a range defined by any of the preceding values. In some embodiments, the cognitive disorder is treatment resistant SCZ.

In one aspect, the present disclosure provides a kit comprising: a. one or more agents to collect a biological sample from a human subject; b. one or more agents to measure a level of CRMP2 and a level of p-CRMP2 from the biological sample obtained from the subject; and c. an instruction to collect the biological sample and measure the levels of CRMP2 and p-CRMP2 using the agents in the kit.

In some embodiments, the agents to measure the levels of CRMP2 and p-CRMP2 are antigen-binding agent. In some embodiments, the antigen-binding agent that recognizes CRMP2 is an anti-CRMP2 antibody; and the antigen-binding agent that recognizes p-CRMP2 is an anti-p-CRMP2 antibody. In some embodiments, the antigen-binding agent that recognizes CRMP2 is an anti-CRMP2 monoclonal antibody; and the antigen-binding agent that recognizes p-CRMP2 is an anti-p-CRMP2 monoclonal antibody. In some embodiments, the anti-p-CRMP2 is an anti-p-Serine 522-CRMP2 (p-S522-CRMP2).

In some embodiments, the biological sample is a blood, saliva, urine, serum, tears, skin, tissue, biopsy, or hair from the subject. In some embodiments, the blood sample comprises peripheral blood mononuclear cells (PBMC). In some embodiments, the PBMC comprises lymphocytes.

In some embodiments, the subject less than 40 years old. In some embodiments, the subject is less than 30 years old. In some embodiments, the subject is greater than 17 years. In some embodiments, the subject is greater than 18 years old.

In some embodiments, the kit is a lateral flow test. In some embodiments, the kit comprises a protein panel, wherein CRMP2 and p-CRMP2 are part of the protein panel.

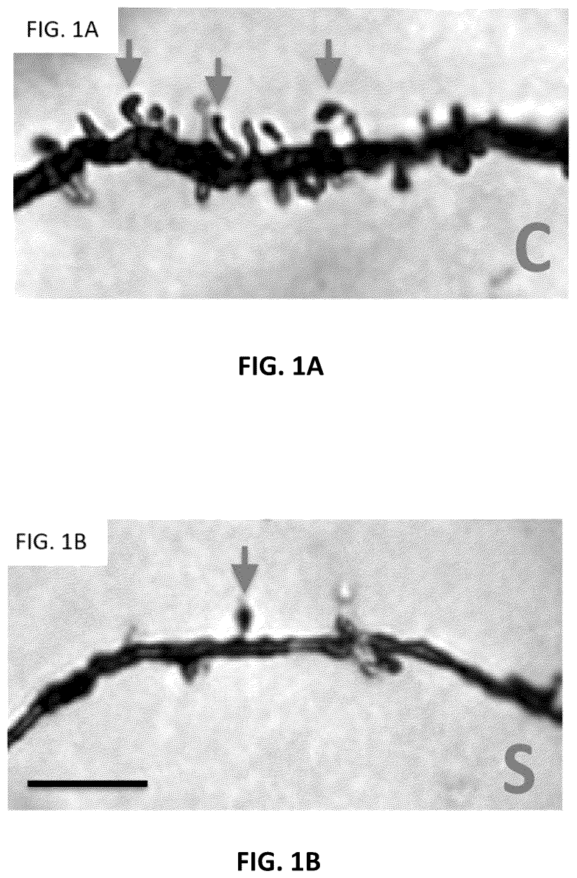

FIGS. 1A-1B show that in brains of patients with SCZ compared with those of unaffected age-matched controls, dendritic spine densities and basilar dendrite lengths are reduced. FIGS. 1A-1B are representative brightfield photomicrographs of the basilar dendrites of Golgi-stained pyramidal neurons in the dorsolateral prefrontal cortex or DLPFC area from a representative control human subject as seen in FIG. 1A compared with a representative patient with SCZ as seen in FIG. 1B. Arrows point to dendritic spines, which are markedly reduced in the SCZ patient. (Scale bar, 5 μm.)

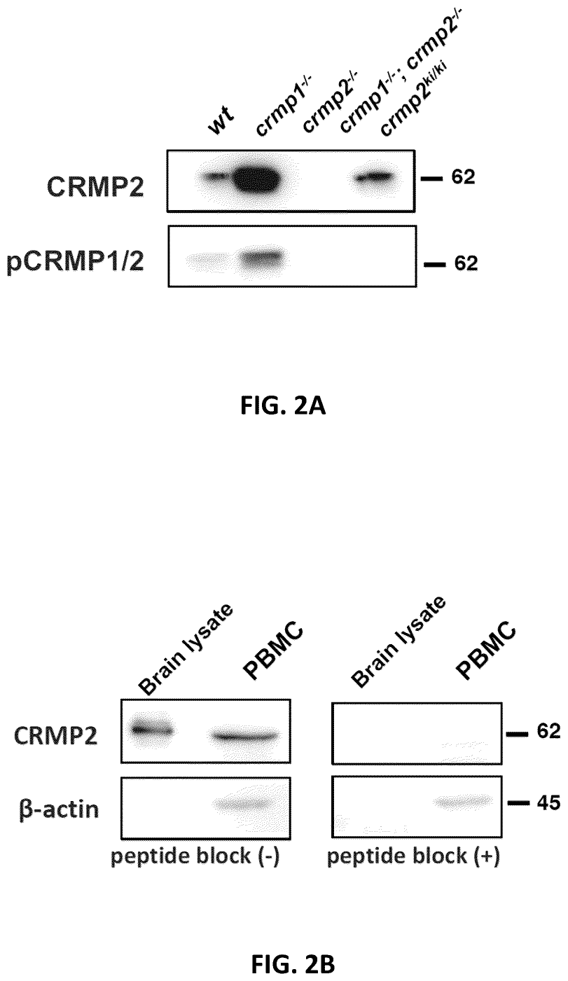

FIGS. 2A-2B show authentication of the sensitivity and specificity of the antibodies against CRMP2 and the phosphorylated form of CRMP2 based on immunoblot analysis. FIG. 2A shows specificity of the anti-CRMP2 monoclonal antibody (9F) [Upper Panel] and the antiphosphorylated CRMP1/2(S522) (p-S522-CRMP2) polyclonal antibody [Lower Panel] demonstrated by immunoblot analysis of the brain lysates from wild type (wt), crmp1−/−, crmp2−/−, crmp1−/−; crmp2−/− and CRMP2S522A knock-in (crmp2ki/ki) mice. In the Upper Panel, a single band of 64 kDa was detected with 9F antibody in brain lysates from wt, crmp1−/− and crmp2ki/ki mice, but was missing in crmp2−/− mice. In the Lower Panel, a single band was detected with anti-p-S522-CRMP2 antibody in brain lysates from wt and crmp1−/−, but not in crmp1−/−; crmp2−/− and crmp2ki/ki mice. FIG. 2B shows a representative immunoblot with anti-CRMP2 antibodies directed against human peripheral blood mononuclear cell (PBMC) fractions from normal healthy control human subjects [Left Panel] compared to positive control brain lysates. In the Right Panel, the bands are seen to be successfully blocked by antigen peptide of CRMP2.

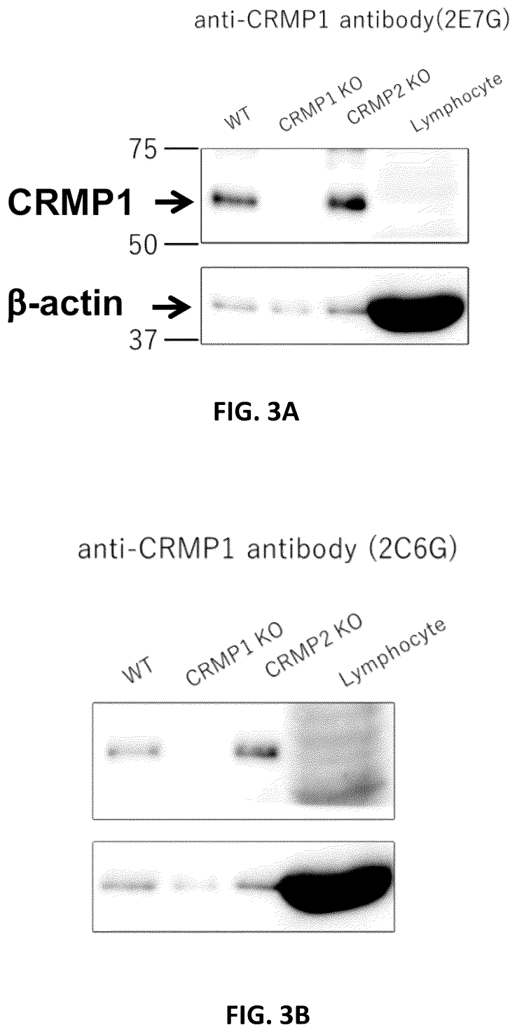

FIGS. 3A-3B show further validation of antibodies used for immunoblotting. The differences seen in SCZ vs. unaffected patients pivoted on differences in the abundance of the CRMP2 isoform rather than of the CRMP1 isoform was proven through the use of antibodies against CRMP1, as well; none of the differences described were related to CRMP1. FIGS. 3A-3B show immunoblot analysis using 2 different anti-CRMP1 antibodies (2E7G in FIG. 3A and 2C6G in FIG. 3B) against wild (WT) and crmp1−/− (CRMP1 KO) or crmp2−/− (CRMP2 KO) mouse brain lysates, as well as human peripheral blood mononuclear cell (PBMC) fractions (specifically, lymphocytes). In brain lysates from wt mice, a single band was detected by 2E7G and 2C6G, but not in the brain lysates from CRMP1-knockout mice, thereby showing the specificity of these anti-CRMP1 antibodies. In this analysis, using these 2 sensitive CRMP1 antibodies, CRMP1 was not detected in human lymphocyte samples.

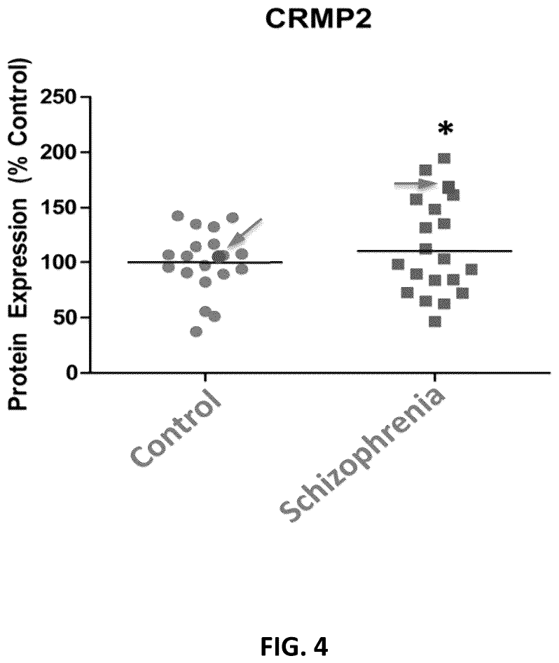

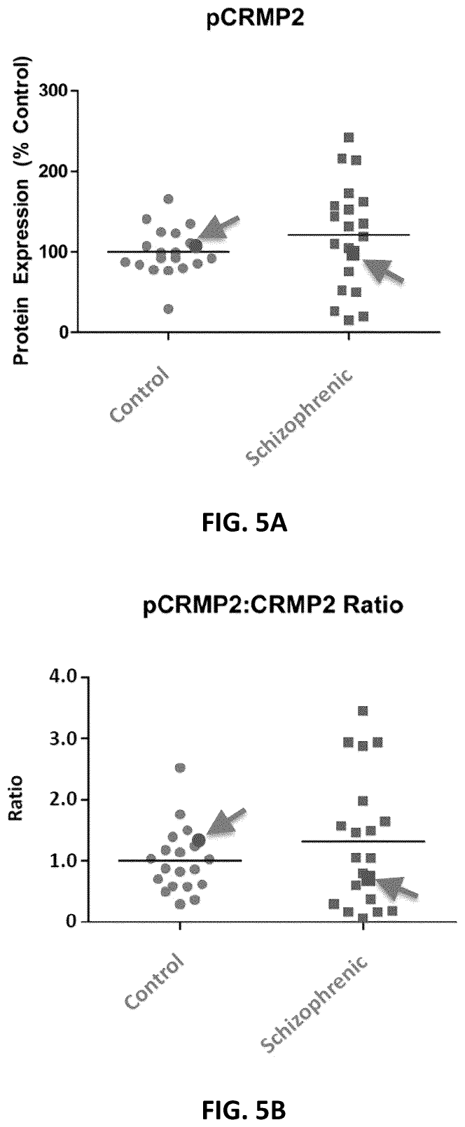

FIG. 4 shows that in brains of patients with SCZ compared with those of unaffected age-matched controls, CRMP2 levels are elevated. Graph depicting the relative protein expression of CRMP2 in control and SCZ patients (whose representative neuropathology is illustrated in FIG. 1B). The SCZ values were normalized to control values (percent control) collected in parallel from the same gel. CRMP2 was significantly increased by 10% in SCZ patients relative to controls (*P=0.05) and CRMP2 protein expression was inversely correlated with basilar dendrite length (r=−0.37, P=0.04). However, as indicated in Table 1, this cohort of brain donors tended to be older (a mean age of ˜60 y old). Focusing on the few patients<40 y-old (among the SCZ patients, indicated by the arrow and square, and among the unaffected controls, indicated by the arrow and dot) suggested exploration of additional CRMP2 parameters—such as diminished p-CRMP2:CRMP2 ratios—as seen in FIGS. 5A-5B, and found to be distinctively low in SCZ patients.

FIGS. 5A-5B show although CRMP2 levels were significantly higher in brains from SCZ patients compared to those from unaffected controls as seen in FIG. 4, the p-CRMP2 protein expression in those SCZ brains in FIG. 5A and, hence, the ratio of p-CRMP2:CRMP2 protein expression in FIG. 5B initially appeared not to be significantly different from controls (p>0.05) when the cohorts were examined in the aggregate. Among the SCZ patients, the brain indicated by the arrow and square came from a 23 year-old female with a CRMP2 level elevated to 129.7 (162% of control, as per FIG. 4) but with a p-CRMP2 level within normal limits (104.5, which is 97.2% of control). While the p-CRMP2:CRMP2 ratio in the unaffected control brain was 1.3 (within normal limits), the ratio in the SCZ brain was quite abnormally low at 0.8 (˜40% lower).

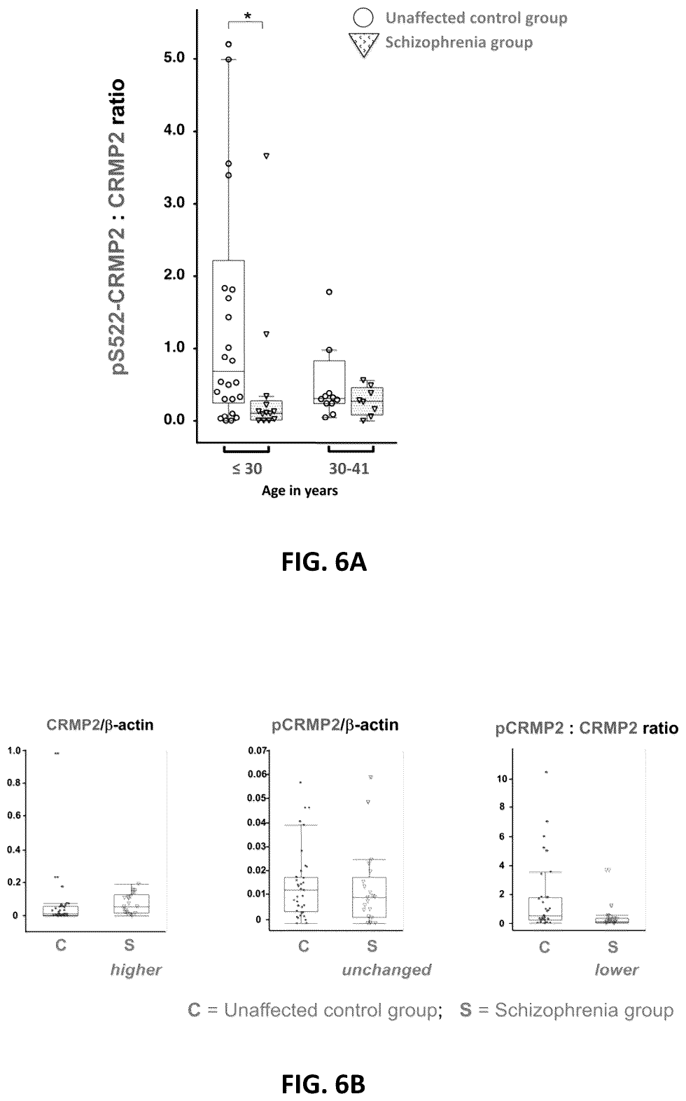

FIGS. 6A-6B show the p-CRMP2:CRMP2 ratio in lymphocytes from patients with SCZ was significantly lower than in lymphocytes from unaffected age-matched control human subjects. CRMP2 phosphorylated at Serine 522 (p-CRMP2) was examined via quantitative Western blots using a specific well-authenticated antibody as seen in FIGS. 5A-5B. FIG. 6A shows that differences were most striking between cohorts that were <30 y old (*P<0.01). FIG. 6B shows that examination (in the aggregate) of the absolute values of CRMP2 (Left) and p-CRMP2 (Center) (using arbitrary values normalized to β-actin as an internal standard) shows that the p-CRMP2:CRMP2 ratio (Right) in human PBMC fractions from patients with SCZ (labeled as S) compared with unaffected controls (labeled as C) was significantly lower (P=0.0051) because the level of CRMP2 (the denominator) was greater (P=0.0146) while pCRMP2 remained unchanged (P=0.4373).

### Definitions

Unless defined otherwise, all terms of art, notations and other technical and scientific terms or terminology used herein are intended to have the same meaning as is commonly understood by one of ordinary skill in the art to which the claimed human subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

As used herein, the term “antibody” or “antigen-binding agent” are intended to cover polyclonal antibodies, multiclonal antibodies, monoclonal antibodies, chimeric antibodies, humanized and primatized antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof antibody fragments such as Fab fragments, F(ab′) fragments, single-chain FvFcs, single-chain Fvs; and derivatives thereof including Fc fusions and other modifications, and any other immunologically active molecule so long as they exhibit the desired biological activity (i.e., antigen association or binding). Moreover, the term further comprises all classes of antibodies (i.e. IgA, IgD, IgE, IgG, and IgM) and all isotypes (i.e., IgG1, IgG2, IgG3, IgG4, IgA1, and IgA2), as well as variations thereof unless otherwise dictated by context.

### Cognitive Disorders

Cognitive disorders are defined as any disorder that impairs the cognitive function of an individual and effects the individual's normal functions to live in society. Patients with cognitive disorders usually require treatment. In some cases, cognitive disorders are results of malfunctions of the brain and nervous systems and often can affect behavior, memory, mental and emotional state of the patients. The damage and degeneration of the nervous system can alter neuronal communication, and these changes can manifest themselves in problems with cognitive function. In other cases, cognitive disorders are caused by other factors such as hormonal imbalances, drug abuse, or lack of proper nutrients during early stage of cognitive development.

In some embodiments, the cognitive disorders comprise any disorders that are related to abnormality of the central nerve system. Examples include but not limited to Alzheimer disease and other dementias, SCZ, traumatic disorders of the nervous system due to head trauma, and cognitive disorders as a result of malnutrition. In some embodiments, the cognitive disorders can occur due to the infection itself or due to an immune response.

In some cases, the cognitive disorders can result in mental disorder or mental illnesses, which are conditions that affect patient's behavior, mood, and ability to think or feel. In some cases, the mental disorders can result in psychotic disorders, which are described as severe mental disorders that causes abnormal thinking and perceptions. Examples include but not limited to SCZ.

The methods and compositions described herein can be used to identify one or more cognitive disorders. The methods and compositions described herein can be used to identify prognosis of one or more cognitive disorders. In one aspect, the method described herein can be used to determine an increased risk for a cognitive disorder in a human subject. In some embodiments, the cognitive disorders also cause psychological symptoms. In some embodiments, the cognitive disorder is dementia, amnesia, delirium, or Schizophrenia. In some embodiments, the cognitive disorder is a mental disorder. In some embodiments, the mental disorder is a psychotic disorder. In some embodiments, the psychotic disorder is Schizophrenia. In some embodiments, the cognitive disorder is SCZ. In some embodiments, the cognitive disorder is not included mood disorder such as Bipolar Disorder.

### Schizophrenia (SCZ)

Schizophrenia (SCZ) is a cognitive disorder due to dysfunctional neuronal networks. SCZ is considered a chronic and severe mental disorder. Patients or human subjects who have SCZ can have combination of symptoms ranging from hallucinations, delusions, disorganized thinking, disorganized speech, extremely disorganized or abnormal motor behavior, or social withdrawal. Some symptoms can vary in type and severity over time, with periods of worsening and remission, while other symptoms can be present. In some cases, patients can experience other symptoms such as depression, anxiety, lack motivation, difficulty in thinking or speaking, neglect of personal hygiene, decreased feelings of enjoyment or pleasure, sudden change in personality and behavior. In some cases, SCZ symptoms can be worsen and improve in cycles known as relapses and remissions. In some cases, SCZ can impair their daily functioning and can be disabling. In some cases, SCZ can result in severe problems and complications such as suicidal thought, depression, anxiety disorder, abuse of alcohol and other drugs, inability to work or maintain daily life, social isolation, and financial problem.

In some cases, patients with SCZ can have positive symptoms, which are characterized by the addition of something not normally present. In some instances, the positive symptoms associated with SCZ comprises sensory changes, hallucinations, and delusions. These positive symptoms can result in psychotic behavior, which suggests lost sense of reality. In other cases, patients with SCZ can have negative symptoms, which are characterized by the absence of something. In some instances, the negative symptoms associated with SCZ comprises emotional and physical disruption, e.g., lack of facial expression, slowed speech patterns, decreased motivation, and withdrawal. In some cases, patients with SCZ can have cognitive impairment, which are characterized by difficulty paying attention, processing information, remembering, and making decisions.

In some embodiments, the human subject who has SCZ can experience positive symptoms of SCZ. In some embodiments, the human subject who has SCZ can further experience negative symptoms of SCZ. In some embodiments, the human subject who has SCZ can further experience cognitive impairment.

In some aspects, the human subject can be diagnosed or suspected of being at high risk for the cognitive disorder. In some embodiments, the human subject is not necessarily diagnosed or suspected of being at high risk for the cognitive disorder. In some embodiments, the human subject is a patient.

In some aspects, SCZ symptoms can be self-identified by the human subject. In some embodiments, SCZ symptoms can be identified by the human subject's family members, friends, relatives, or those who know the human subject. In some embodiments, SCZ symptoms can be identified by a healthcare provider or clinical investigator.

In some aspects, the human subject who has SCZ can have one or more symptoms related to SCZ. In some embodiments, the human subject who has SCZ may have two or more, three or more, four or more, or five or more symptoms related to SCZ.

In some aspects, the human subject who has SCZ can have had SCZ symptoms for at least 1 day, 1 week, 1 month, or 1 year.

### Patient Demography

In some aspects, male human subjects can develop the cognitive symptoms in their late teens or in the early to mid-20's. In some embodiments, the male human subjects can be diagnosed with the cognitive disorder in their late teens or in the early to mid-20's. In another aspects, female human subjects can develop the cognitive symptoms in early 20's to 30's. In some embodiments, the female human subjects can be diagnosed with the cognitive disorder in early 20's to 30's. In some embodiments, children under age of 15 are not diagnosed with the cognitive disorder. In other embodiments, adult older than age of 50 are not diagnosed with the cognitive disorder. In some embodiments, the cognitive disorders cause psychological symptoms. In some embodiments, the cognitive disorder is a mental disorder. In some embodiments, the mental disorder is a psychotic disorder. In some embodiments, the psychotic disorder is SCZ. In some embodiments, the cognitive disorder is SCZ. In some embodiments, the SCZ is treatment resistant SCZ.

In some cases, a patient population that are diagnosed with the methods described herein can be in their late teens or in the early 20's. In some aspects, a human subject who is diagnosed with the cognitive disorder is a male human. In some embodiments, the human subject who is diagnosed with the cognitive disorder is a female human. In some embodiments, the human subject is a young adult. In some aspects, a human subject who is diagnosed with the cognitive disorder identifies as a man, woman, or nonbinary. In some embodiments, the cognitive disorders cause psychological symptoms. In some embodiments, the cognitive disorder is a mental disorder. In some embodiments, the mental disorder is a psychotic disorder. In some embodiments, the psychotic disorder is Schizophrenia. In some embodiments, the cognitive disorder is SCZ. In some embodiments, the SCZ is treatment resistant SCZ.

In various embodiments, the human subject is a male human. In some aspects, the human subject identifies as a man, woman, or nonbinary. In some embodiments, the human subject is less than 60 years old. In some embodiments, the human subject is less than 50 years old. In some embodiments, the human subject is less than 40 years old. In some embodiments, the human subject is less than 30 years old. In various embodiments, the human subject is about 17 years old to about 60 years old. In some embodiments, the human subject is about 17 years old to about 50 years old. In some embodiments, the human subject is about 17 years old to about 40 years old. In some embodiments, the human subject is about 17 years old to about 30 years old. In various embodiments, the human subject is about 18 years old to about 60 years old. In some embodiments, the human subject is about 18 years old to about 50 years old. In some embodiments, the human subject is about 18 years old to about 40 years old. In some embodiments, the human subject is about 18 years old to about 30 years old. In various embodiments, the human subject is at least 15 years old. In some embodiments, the human subject is at least 17 years old. In some embodiments, the human subject is at least 18 years old. In some embodiments, the human subject is at least 20 years old. In some embodiments, the human subject is at least 25 years old.

In various embodiments, the human subject experiences one or more symptoms of the cognitive disorder comprises delusion, hallucination, disorganized thinking, disorganized speech, extremely disorganized or abnormal motor behavior, or social withdrawal.

In some cases, the human subject can be at risk for one or more of the cognitive disorders. In some embodiments, the human subject can have factors that contribute to risk for the cognitive disorder, e.g., known genetic variants or mutations, family history (first-degree relatives), stressful life experience, history of abuse, known chemical imbalance in the brain, traumatic brain injury, or history of drug abuse. In some embodiments, the human subject can be diagnosed with an increased risk for the cognitive disorder and not have any discernible symptoms. In some embodiments, the cognitive disorders cause psychological symptoms. In some embodiments, the cognitive disorder is a mental disorder. In some embodiments, the mental disorder is a psychotic disorder. In some embodiments, the psychotic disorder is SCZ. In some embodiments, the cognitive disorder is SCZ. In some embodiments, the SCZ is treatment resistant SCZ.

### Diagnostic Criteria

Current diagnosis of the cognitive disorders such as SCZ relies on the analysis of a patient's symptoms based on information obtained from physical examination, patient's family history, emotional history, medical evaluation, and mental status examination. Healthcare provider uses criteria and guideline based on the Diagnostic and Statistical Manual of Mental Disorders (DSM-5). According to the DSM-5, a SCZ diagnosis requires the following: i) at least two of five main symptoms, which are delusions, hallucinations, disorganized or incoherent speaking, disorganized or unusual movements and negative symptoms; ii) duration of symptoms and effects, which must persist for at least 6 months and within this 6 month period must include at least 1 month of active-phase symptoms; and iii) social or occupational dysfunction, which symptoms disrupt the ability to work or relationship. In some aspects, correct diagnosis of the cognitive disorder such as SCZ can be difficult since it is human subjective and can be confused with other medical/cognitive conditions. In some embodiments, a biological sample can be obtained from a human subject for the methods described herein when diagnosis of the cognitive disorder such as SCZ is uncertain.

In some aspects, medical information can be obtained from the human subject to diagnose the cognitive disorder. In some embodiments, the biological sample can be obtained from the human subject for the methods described herein prior to obtaining medical information related to the cognitive disorder. In some embodiments, the biological sample can be obtained from the human subject for the methods described herein after obtaining medical information related to the cognitive disorder. In some embodiments, the methods described herein can be performed after a consultation with a healthcare provider. In some embodiments, the methods described herein can be performed by the healthcare provider. In some embodiments, the methods described herein can be performed by non-healthcare providers. In some embodiments, the methods described herein can be performed by a clinical investigator. In some embodiments, the cognitive disorders cause psychological symptoms. In some embodiments, the cognitive disorder is a mental disorder. In some embodiments, the mental disorder is a psychotic disorder. In some embodiments, the psychotic disorder is SCZ. In some embodiments, the cognitive disorder is SCZ. In some embodiments, the SCZ is treatment resistant SCZ.

### Biological Samples

As used herein, the terms “collecting a biological sample” includes collecting or obtaining a sample directly or indirectly. In some embodiments, the sample is taken from the human subject by the same party (e.g. a testing laboratory) that subsequently determines levels of CRMP2 and/or p-CRMP2 from the biological sample. In some embodiments, the sample is received (e.g. by a testing laboratory) from another entity that collected the sample from the subject (e.g. a physician, nurse, healthcare provider, phlebotomist, clinical investigator or medical caregiver). In some embodiments, the sample is taken from the subject by a medical professional under direction of a separate entity (e.g. a testing laboratory) and subsequently provide to the entity (e.g. the testing laboratory). In some embodiments, the sample is taken by the subject or the subject's caregiver at home and subsequently provided to the party that determines levels of CRMP2 and/or p-CRMP2 from the sample (e.g. a testing laboratory).

In some embodiments, the biological sample can be obtained from the human subject to use for the methods described herein. In various embodiments, the biological sample obtained from the human subject can be blood, saliva, urine, serum, tears, skin, tissue, or hair. In various embodiments, the biological sample obtained from the human subject is blood, saliva, or urine. In some embodiments, the blood sample comprises peripheral blood mononuclear cells (PBMC). In some embodiments, the PBMC comprises lymphocytes. In some embodiments, the biological sample obtained from the human subject can comprise cells or tissues that express Collapsin response mediator protein 2 (CRMP2).

CRMP2 and p-CRMP2

Collapsin response mediator protein 2 (CRMP2), which is also known as Dihydropyrimidinase-like 2 (DPYSL2), is a master regulator of axon guidance, dendritic branching, and spine formation. CRMP2 is known to be a neural network modulator and a master regulator of cytoskeleton. The CRMP family of proteins are now known to consist of five homologous cytosolic proteins, CRMP1 through CRMP5. CRMP form homo- and hetero-tetramers and facilitate neuron guidance, growth and polarity. CRMP2 actively binds cytoskeletal elements in its nonphosphorylated active state. Phosphorylation of CRMP2 (p-CRMP2), which is a two-step process, inactivates it and induces it to release cytoskeletal elements. Cdk5 first phosphorylates CRMP2 at Ser522, priming it for glycogen synthase kinase 3β (GSKβ) to phosphorylate it at Thr514 and Ser518. There is a homeostasis between inactive (phosphorylated) and active (nonphosphorylated) CRMP2, which is an ongoing physiologic adaptive mechanism for preventing abnormal neuronal sprouting. Dendritic spines are the point of contact for interneuronal synaptic communication. Nonphosphorylated CRMP2 is expressed throughout the neuron, including the dendritic spines; phosphorylated CRMP2 is not expressed in the spines, suggesting that when CRMP2 becomes inactivated, it leaves or is excluded from the spines. Agents known to decrease inactivated CRMP2 (e.g., lithium) also increase dendritic spine volume and density, an action abrogated by the elimination of CRMP2.

Measuring Levels of CRMP2 and/or p-CRMP2

In one aspect, the level of CRMP2 can be measured from the biological sample obtained from the human subject using various techniques known in the art. In some embodiments, the level of CRMP2 can be measured by an assay comprising a proteomic assay. In some embodiments, the proteomic assay is immunoassay, mass spectrometry, or intracellular flow cytometry. In some embodiments, the immunoassay comprises western blotting, dot blotting, quantitative enzyme-linked immunosorbent assays (ELISA), immunocytochemistry (ICC), immunohistochemistry (IHC), protein multiplex assay, or lateral flow test. In some embodiments, the assay is a lateral flow test.

In another aspects, the levels of CRMP2 and p-CRMP2 can be measured from the biological sample obtained from the human subject using various techniques known in the art. In some embodiments, the levels of CRMP2 and p-CRMP2 can be measured by an assay comprising a proteomic assay. In some embodiments, the proteomic assay is immunoassay, mass spectrometry, or intracellular flow cytometry. In some embodiments, the immunoassay comprises western blotting, dot blotting, quantitative enzyme-linked immunosorbent assays (ELISA), immunocytochemistry (ICC), immunohistochemistry (IHC), protein multiplex assay, or lateral flow test. In some embodiments, the assay is a lateral flow test.

In some embodiments, the proteomic assay is performed using the biological sample obtained from the human subject and the level of CRMP2 is detected from results obtained from the proteomic assay. In some embodiments, the proteomic assay is performed using the biological sample obtained from the human subject and the levels of CRMP2 and p-CRMP2 are detected from results obtained from the proteomic assay.

In some embodiments, the assay comprises an agent. In various embodiments, the agent is an antigen-binding agent. In some embodiments, the antigen-binding agent is an antibody. In some embodiments, the antigen-binding agent that recognizes CRMP2 is an anti-CRMP2 antibody and the antigen-binding agent that recognizes p-CRMP2 is an anti-p-CRMP2 antibody. In some embodiments, the antigen-binding agent that recognizes CRMP2 is an anti-CRMP2 monoclonal antibody and the antigen-binding agent that recognizes p-CRMP2 is an anti-p-CRMP2 monoclonal antibody. In some embodiments, the anti-p-CRMP2 antibody recognizes p-CRMP2 at Serine 522 (p-S522-CRMP2).

In some embodiments, the agent to measure the levels of CRMP2 is antigen-binding agent. In some embodiments, the antigen-binding agent to measure the level of CRMP2 is an antibody. In some embodiments, the antigen-binding agent that recognizes CMRP2 is an anti-CRMP2 antibody. In some embodiments, the antigen-binding agent that recognizes CRMP2 is an anti-CRMP2 monoclonal antibody. In some embodiments, the agents to measure the levels of CRMP2 and p-CRMP2 are antigen-binding agents. In some embodiments, the antigen-binding agents to measure the levels of CRMP2 and p-CRMP2 are antibodies. In some embodiments, the antigen-binding agent that recognizes CRMP2 is an anti-CRMP2 antibody and the antigen-binding agent that recognizes p-CRMP2 is an anti-p-CRMP2 antibody. In some embodiments, the antigen-binding agent that recognizes CRMP2 is an anti-CRMP2 monoclonal antibody and the antigen-binding agent that recognizes p-CRMP2 is an anti-p-CRMP2 monoclonal antibody. In some embodiments, the anti-p-CRMP2 antibody recognizes p-CRMP2 at Serine 522 (p-S522-CRMP2). In some embodiments, the antibodies comprise an anti-CRMP2 and an anti-p-CRMP2. In some embodiments, the anti-p-CRMP2 is an anti-p-Serine 522-CRMP2 (p-S522-CRMP2).

In some embodiments, the assay comprises a protein panel in which CRMP2 and p-CRMP2 are part of the protein panel. In some embodiments, CRMP2 can be one protein in a panel of protein that is detected. In some embodiments, p-CRMP2 can be one protein in a panel of protein that is detected. In some embodiments, both CRMP2 and p-CRMP2 can be proteins in a panel of proteins that are detected.

In various embodiments, the CRMP2 is phosphorylated at Serine 522 (p-S522-CRMP2). In some embodiments, measuring the level of p-CRMP2 comprises measuring a phosphorylation of CRMP2 at Serine 522.

In some embodiments, the biological sample obtained from the human subject can be tested for CRMP2, p-CRMP2, or functional analogue thereof. In some embodiments, the biological sample obtained from the human subject can be tested for a post-translational modification in the CRMP2. In some embodiments, the post-translational modification can be phosphorylation, acetylation, glycosylation, or other post-translational modification known by a person of skill in the art. In some embodiments, additional post-translational modification can be tested for both CRMP2 and p-S522-CRMP2.

In some embodiments, the human subject can be retested the level of CRMP2 over a period of at least 1 day, 1 week, 1 month, 1 year, 5 years, or 10 years. In some embodiments, the healthcare provider or clinical investigator can continue monitoring the level of CRMP2 from the human subject over a period of at least 1 day, 1 week, 1 month, 1 year, 5 years, or 10 years.

In some embodiments, the human subject can be retested the levels of p-CRMP2 and CRMP2 over a period of at least 1 day, 1 week, 1 month, 1 year, 5 years, or 10 years. In some embodiments, the healthcare provider or clinical investigator can continue monitoring the levels of CRMP2 and p-CRMP2 from the human subject over a period of at least 1 day, 1 week, 1 month, 1 year, 5 years, or 10 years.

In some embodiments, the biological sample can be obtained from the human subject to use for detecting levels of CRMP2. In some embodiments, the biological sample can be obtained from the human subject to use for detecting levels of CRMP2 and p-CRMP2. In some embodiments, the biological sample obtained from the human subject can be blood, saliva, urine, serum, tears, skin, tissue, or hair. In some embodiments, the biological sample obtained from the human subject is blood, saliva, or urine. In some embodiments, the blood sample comprises peripheral blood mononuclear cells (PBMC). In some embodiments, the PBMC comprises lymphocytes. In some embodiments, the biological sample obtained from the human subject can comprise cells or tissues that express CRMP2.

In some embodiments, the patient's sample used for CRMP2 detection is blood sample. In some embodiments, the patient's sample used for CRMP2 and p-CRMP2 detection is blood sample.

In some embodiments, determining whether the human subject has or is predisposed to a cognitive disorder such as SCZ is based on analysis of the human subject's symptoms, which rely on information obtained from physical examination, family history, emotional history, medical evaluation, and mental status examination. Methods described herein for determining an increase risk for the cognitive disorder such as SCZ, in some embodiments, are improved as compared to methods comprising cognitive tests, physical examination, family history, emotional history, medical evaluation, and mental status examination. In some embodiments, methods described herein for determining an increased risk for the cognitive disorder such as SCZ provide an improvement of the cognitive disorder diagnostic method.

Computing Ratio of Phosphorylated CRMP2 to CRMP2 (p-CRMP2:CRMP2)

The ratio of the phosphorylated CRMP2 (p-CRMP2) at Serine 522 (Ser522 or S522) to the nonphosphorylated CRMP2 (p-CRMP2:CRMP2 ratio) can be computed to generate diagnostic criteria for SCZ. Samples from SCZ patients show to have higher level of CRMP2 and lower ratio of p-CRMP2:CRMP2 when compared to the sample from an unaffected age-matched human subjects.

As used herein, the terms “control” or “control sample” or “control subject” or “control group” refer to subjects or biological samples obtained from subjects or a group of subjects known not to have cognitive disorders. In some instances, the control group of subjects is known not to have SCZ.

As used herein, the terms “reference” or “reference level” or “reference ratio” refer to the data or value obtained from subject or a group of subjects known not to have cognitive disorders. In some instances, the group of subjects is known not to have SCZ. In some embodiments, the data or value obtained as reference level is from the control group.

If the human subject has the ratio of p-CRMP2:CRMP2 below 1.0, 0.9, 0.8, 0.7, 0.6, 0.5, 0.4, 0.3, 0.2, 0.1 or within a range defined by any of the preceding values, the human subject is considered to have added risk of having a cognitive disorder such as SCZ. In this case, the human subject can have one or more symptoms of the cognitive disorder such as SCZ.

In some embodiments, the ratio of p-CRMP2:CRMP2 can be computed on an electronic device. In some embodiments, the ratio of p-CRMP2:CRMP2 can be computed by color indicator upon detection of CRMP2 and p-CRMP2.

In various embodiments, the method further comprises identifying the human subject with low to high risk of having the cognitive disorder if the p-CRMP2:CRMP2 ratio of the subject is decreased compared to the reference p-CRMP2:CRMP2 ratio. In some embodiments, the method further comprises identifying the human subject with added risk of having the cognitive disorder if the p-CRMP2:CRMP2 ratio of the human subject is lower than 1.0, 0.9, 0.8, 0.7, 0.6, 0.5, 0.4, 0.3, 0.2, 0.1 or within a range defined by any of the preceding values. In some embodiments, the method further comprises identifying the human subject with added risk of having the cognitive disorder if the p-CRMP2:CRMP2 ratio of the human subject is between 0.1 to 0.4. In some embodiments, the method further comprises identifying the human subject with added risk of having the cognitive disorder if the p-CRMP2:CRMP2 ratio of the human subject is between 0.2 to 0.5. In some embodiments, the method further comprises identifying the human subject with added risk of having the cognitive disorder if the p-CRMP2:CRMP2 ratio of the human subject is between 0.1 to 1.0. In some embodiments, the method further comprises identifying the human subject with added risk of having the cognitive disorder if the p-CRMP2:CRMP2 ratio of the human subject is about 40% lower than the reference p-CRMP2:CRMP2 ratio. In some embodiments, the method further comprises identifying the human subject with added risk of having the cognitive disorder if the p-CRMP2:CRMP2 ratio of the human subject is about 10% lower than the reference p-CRMP2:CRMP2 ratio. In some embodiments, the method further comprises identifying the human subject with added risk of having the cognitive disorder if the p-CRMP2:CRMP2 ratio of the human subject is within a range of from 10% lower to 40% lower than the reference p-CRMP2:CRMP2 ratio. In some embodiments, the cognitive disorder is SCZ. In some embodiments, the SCZ is treatment resistant SCZ.

In various embodiments, the human subject with high added risk of having the cognitive disorder is further identified with one or more severe symptoms of the cognitive disorder. In some embodiments, the human subject with moderate added risk of having the cognitive disorder is further identified with one or more moderate symptoms of the cognitive disorder. In some embodiments, the human subject with low added risk of having the cognitive disorder is further identified with one or more mild symptoms of the cognitive disorder. In some embodiments, the cognitive disorders cause psychological symptoms. In some embodiments, the cognitive disorder is a mental disorder. In some embodiments, the mental disorder is a psychotic disorder. In some embodiments, the psychotic disorder is SCZ. In some embodiments, the cognitive disorder is SCZ. In some embodiments, the SCZ is treatment resistant SCZ.

In some embodiments, the human subject can retest the levels of CRMP2 and p-CRMP2 and the ratio of p-CRMP2:CRMP2 over a period of at least 1 day, 1 week, 1 month, 1 year, 5 years, or 10 years. In some embodiments, the healthcare provider or clinical investigator can continue monitoring the levels of CRMP2 and p-CRMP2 and the ratio of p-CRMP2:CRMP2 from the human subject over a period of at least 1 day, 1 week, 1 month, 1 year, 5 years, or 10 years. In some embodiments, if the human subject has the ratio of p-CRMP2:CRMP2 lower than 1.0, healthcare provider or clinical investigator can continue monitoring the ratio of p-CRMP2:CRMP2 over a period of at least 1 day, 1 week, 1 month, 1 year, 5 years, or 10 years. In some embodiments, if the human subject has the ratio of p-CRMP2:CRMP2 lower than 1.0, healthcare provider or clinical investigator can continue monitoring the ratio of p-CRMP2:CRMP2 over a period of at least 1 day, 1 week, 1 month, 1 year, 5 years, or 10 years. In some embodiments, if the human subject has the ratio of p-CRMP2:CRMP2 more than 1.0, the healthcare provider or clinical investigator can continue monitoring the ratio of p-CRMP2:CRMP2 over a period of at least 1 day, 1 week, 1 month, 1 year, 5 years, or 10 years.

In one aspect, the present disclosure provides a method for determining an increased risk for a cognitive disorder in a human subject, comprising: collecting a biological sample from the human subject; determining, by an assay, a level of collapsing response mediator protein-2 (CRMP2) in the biological sample, wherein the assay comprises: i. contacting the biological sample with an agent that recognizes CRMP2, and ii. measuring the level of bound CRMP2 and thereby determining the level of CRMP2 present in the biological sample; and comparing the level of CRMP2 to a reference level of CRMP2, wherein the reference level of CRMP2 is derived from a group of subjects known not to have the cognitive disorder, and wherein the human subject is identified with the increased risk for the cognitive disorder if the level of CRMP2 is increased when compared to the reference level of CRMP2. In some embodiments, the assay further comprises: i. contacting the biological sample with an agent that recognizes phosphorylated CRMP2 (p-CRMP2), and ii. measuring a level of bound p-CRMP2 and thereby determining the level of p-CRMP2 present in the biological sample.

In various embodiments, the method further comprises a. computing a ratio of the p-CRMP2 to CRMP2 (p-CRMP2:CRMP2 ratio); and b. comparing the p-CRMP2:CRMP2 ratio to a reference p-CRMP2:CRMP2 ratio; wherein the reference p-CRMP2:CRMP2 ratio is derived from a second group of subjects known not to have the cognitive disorder.

In various embodiments, the cognitive disorder is not age-related. In some embodiments, the cognitive disorder is SCZ. In some embodiments, the SCZ is an early stage of SCZ. In some embodiments, the SCZ is treatment resistant SCZ.

### Additional Tests

In some aspects, in addition to measuring the levels of p-CRMP2 and CRMP2 in a human subject, clinical investigators can administer a Clinical Assessment Interview for Negative Symptoms (CAINS), Brief Negative Symptom Scale (BNSS), Positive and Negative Symptoms Scale (PANSS), Scale for the Assessment of Positive Symptoms (SAPS), Scale for the Assessment of Negative Symptoms (SANS), Negative Symptom Assessment-16 (NSA-16), Clinical Global Impression Schizophrenia (CGI-SCH) or combination of two or more of the preceding to the human subject. In some aspects, in addition to measuring the levels of p-CRMP2 and CRMP2 in a human subject, clinical investigators can perform a PANSS to assess the presence of positive symptoms, negative symptoms and general psychopathology of the human subject. In some embodiments, the human subject has a cognitive disorder. In some embodiments, the cognitive disorder is a mental disorder. In some embodiments, the mental disorder is a psychotic disorder. In some embodiments, the psychotic disorder is Schizophrenia. In some embodiments, the cognitive disorder is Schizophrenia.

In some aspects, in addition to measuring the levels of p-CRMP2 and CRMP2 in a human subject, clinical investigators can perform a Clinical Assessment Interview for Negative Symptoms (CAINS), Brief Negative Symptom Scale (BNSS), Positive and Negative Symptoms Scale (PANSS), Scale for the Assessment of Positive Symptoms (SAPS), Scale for the Assessment of Negative Symptoms (SANS), Negative Symptom Assessment-16 (NSA-16), Clinical Global Impression Schizophrenia (CGI-SCH) or combination of two or more of the preceding with the human subject before and/or after receiving a therapeutic agent or clinical investigational product. In some embodiments, the therapeutic agent or clinical investigational product is a DAAO inhibitor, VMAT2 inhibitor, or selective muscarinic M4 agonist. In some embodiments, the DAAO inhibitor is Luvadaxistat. In some embodiments, the therapeutic agent or clinical investigational product is a modulator of serotonin 5-HT_{2A}. In some embodiments, the modulator of serotonin 5-HT_{2A} is Pimavanserin. In some embodiments, the therapeutic agent or clinical investigational product is Pimavanserin, Luvadaxistat, Valbenazine, Tetrabenazine, Deutetrabenazine or NBI-1117568. In some embodiments, the VMAT2 inhibitor is Valbenazine, Tetrabenazine or Deutetrabenazine. In some embodiments, the selective muscarinic M4 agonist is NBI-1117568. In some embodiments, the human subject has a cognitive disorder. In some embodiments, the cognitive disorder is a mental disorder. In some embodiments, the mental disorder is a psychotic disorder. In some embodiments, the psychotic disorder is Schizophrenia. In some embodiments, the cognitive disorder is Schizophrenia.

In various embodiments, the present disclosure provides a method of testing CRMP2 level from a blood sample obtained from a human subject. In some embodiments, the blood sample comprises PBMC or lymphocytes. In some embodiments, the present disclosure provides a method of testing CRMP2 and p-CRMP2 levels from a blood sample. In some embodiments, the blood sample comprises PBMC or lymphocytes. In some embodiments, the method further comprises performing a CAINS, BNSS, PANSS, SAPS, SANS, NSA-16, CGI-SCH or combination of two or more of the preceding with the human subject. In some aspects, the method further comprises performing a PANS S to assess the presence of positive symptoms, negative symptoms and general psychopathology of the human subject. In some embodiments, the method further comprises administering a therapeutic agent or clinical investigational product to the human subject. In some embodiments, the therapeutic agent or clinical investigational product is a DAAO inhibitor, VMAT2 inhibitor, or selective muscarinic M4 agonist. In some embodiments, the DAAO inhibitor is Luvadaxistat. In some embodiments, the VMAT2 inhibitor is Valbenazine, Tetrabenazine or Deutetrabenazine. In some embodiments, the selective muscarinic M4 agonist is NBI-1117568. In some embodiments, the therapeutic agent or clinical investigational product is a modulator of serotonin 5-HT_{2A}. In some embodiments, the modulator of serotonin 5-HT_{2A} is Pimavanserin. In some embodiments, the therapeutic agent or clinical investigational product is Pimavanserin, Luvadaxistat, Valbenazine, Tetrabenazine, Deutetrabenazine or NBI-1117568. In some embodiments, the human subject has a cognitive disorder. In some embodiments, the cognitive disorder is a mental disorder. In some embodiments, the mental disorder is a psychotic disorder. In some embodiments, the psychotic disorder is Schizophrenia. In some embodiments, the cognitive disorder is Schizophrenia. In some embodiments, the Schizophrenia is treatment resistant Schizophrenia.

In various aspects, a method as disclosed and described herein further comprises performing a CAINS, BNSS, PANSS, SAPS, SANS, NSA-16, CGI-SCH or combination of two or more of the preceding with the human subject. In some aspects, the further comprises performing a PANS S to assess the presence of positive symptoms, negative symptoms and general psychopathology of the human subject. In some embodiments, a method as disclosed and described herein further comprises administering a therapeutic agent or clinical investigational product to the human subject. In some embodiments, the therapeutic agent or clinical investigational product is a DAAO inhibitor, VMAT2 inhibitor, or selective muscarinic M4 agonist. In some embodiments, the DAAO inhibitor is Luvadaxistat. In some embodiments, the VMAT2 inhibitor is Valbenazine, Tetrabenazine or Deutetrabenazine. In some embodiments, the selective muscarinic M4 agonist is NBI-1117568. In some embodiments, the therapeutic agent or clinical investigational product is a modulator of serotonin 5-HT_{2A}. In some embodiments, the modulator of serotonin 5-HT_{2A} is Pimavanserin. In some embodiments, the therapeutic agent or clinical investigational product is Pimavanserin, Luvadaxistat, Valbenazine, Tetrabenazine, Deutetrabenazine or NBI-1117568. In some embodiments, the cognitive disorder is a mental disorder. In some embodiments, the mental disorder is a psychotic disorder. In some embodiments, the psychotic disorder is Schizophrenia. In some embodiments, the cognitive disorder is Schizophrenia.

In some embodiments, the human subject can be tested for other biomarkers associated with the cognitive disorders. In some embodiments, the healthcare provider or clinical investigator can order additional blood tests to detect other biomarkers associated with the cognitive disorders. In some embodiments, the method further comprises sequencing a genome of the human subject. In some embodiments, the method further comprises testing DNA, mRNA, cDNA, or protein of the human subject to detect other biomarkers associated with the cognitive disorder.

In various embodiments, the assay comprises a protein panel, wherein CRMP2 and p-CRMP2 are part of the protein panel. In some embodiments, the human subject is tested for at least one other protein in addition to CRMP2. In some embodiments, the human subject is tested for at least one other protein in addition to CRMP2 and p-CRMP2. In some embodiments, the human subject is tested for at least two other proteins in addition to CRMP2. In some embodiments, the human subject is tested for at least two other proteins in addition to CRMP2 and p-CRMP2.

In various embodiments, the method further comprises sequencing a genome of the human subject. In some embodiments, the method further comprises testing DNA, mRNA, or cDNA of the subject to detect other biomarkers. In some embodiments, the method further comprises monitoring the levels of CRMP2 and p-CRMP2 from the human subject over a period of at least 1 year.

In some embodiments, the method further comprises retesting the levels of CRMP2 and p-CRMP2 from the subject if the p-CRMP2:CRMP2 ratio of the human subject is lower than 1.0. In some embodiments, the method further comprises retesting the levels of CRMP2 and p-CRMP2 from the subject if the p-CRMP2:CRMP2 ratio of the human subject is lower than 0.9. In some embodiments, the method further comprises retesting the levels of CRMP2 and p-CRMP2 from the subject if the p-CRMP2:CRMP2 ratio of the human subject is lower than 0.8. In some embodiments, the method further comprises retesting the levels of CRMP2 and p-CRMP2 from the subject if the p-CRMP2:CRMP2 ratio of the human subject is lower than 0.7. In some embodiments, the method further comprises retesting the levels of CRMP2 and p-CRMP2 from the subject if the p-CRMP2:CRMP2 ratio of the human subject is lower than 0.6. In some embodiments, the method further comprises retesting the levels of CRMP2 and p-CRMP2 from the subject if the p-CRMP2:CRMP2 ratio of the human subject is lower than 0.5. In some embodiments, the method further comprises retesting the levels of CRMP2 and p-CRMP2 from the subject if the p-CRMP2:CRMP2 ratio of the human subject is lower than 0.4. In some embodiments, the method further comprises retesting the levels of CRMP2 and p-CRMP2 from the subject if the p-CRMP2:CRMP2 ratio of the human subject is lower than 0.3. In some embodiments, the method further comprises retesting the levels of CRMP2 and p-CRMP2 from the subject if the p-CRMP2:CRMP2 ratio of the human subject is lower than 0.2. In some embodiments, the method further comprises retesting the levels of CRMP2 and p-CRMP2 from the subject if the p-CRMP2:CRMP2 ratio of the human subject is lower than 0.1. In some embodiments, the method further comprises performing a CAINS, BNSS, PANSS, SAPS, SANS, NSA-16, CGI-SCH or combination of two or more of the preceding with the human subject. In some aspects, the method further comprises performing a PANSS to assess the presence of positive symptoms, negative symptoms and general psychopathology of the human subject.

In some embodiments, the cognitive disorders cause psychological symptoms. In some embodiments, the cognitive disorder is a mental disorder. In some embodiments, the mental disorder is a psychotic disorder. In some embodiments, the psychotic disorder is Schizophrenia. In some embodiments, the cognitive disorder is Schizophrenia. In various embodiments, the human subject can be tested for other biomarkers associated with SCZ. In some embodiments, the healthcare provider can order additional blood tests to detect other biomarkers associated with SCZ. In some embodiments, the method further comprises sequencing a genome of the human subject. In some embodiments, the method further comprises testing DNA, mRNA, cDNA, or protein of the human subject to detect other biomarkers associated with SCZ. In some embodiments, other biomarkers for SCZ are described in, for example, U.S. Pat. No. 8,258,284 B2, which is incorporated by reference in its entirety.

In some embodiments, the human subject can have more than one cognitive disorder. In some embodiments, the cognitive disorder comprises SCZ. In some embodiments, the cognitive disorder is SCZ. In some embodiments, the human subject may have just one cognitive disorder. In some embodiments, the human subject can be identified with the cognitive disorder and at least one additional cognitive disorder. In some embodiments, the human subject can be identified with SCZ and at least one additional cognitive disorder.

### Therapeutics

In another aspect, the present disclosure provides a method comprising: collecting a biological sample from the human subject; determining, by an assay, a level of CRMP2 in the biological sample from the subject, wherein the assay comprises: i. contacting the biological sample with an agent that recognizes CRMP2, ii. measuring the level of bound CRMP2 and thereby determining the level of CRMP2 present in the biological sample, and iii. computing the risk of the human subject having a cognitive disorder based on comparing the level of CRMP2 to a reference level of CRMP2; and administering a therapeutic agent or clinical investigational product to the human subject, wherein the therapeutic agent or clinical investigational product is configured to mitigate or alleviate one or more symptoms of the cognitive disorder in the human subject. In some embodiments, the therapeutic agent or clinical investigational product is configured to modulate the level of CRMP2. In some embodiments, the therapeutic agent or clinical investigational product is configured to modulate the p-CRMP2:CRMP2 ratio.

In various embodiments, the assay further comprises: contacting the biological sample with an agent that recognizes phosphorylated CRMP2 (p-CRMP2), and measuring a level of bound p-CRMP2 and thereby determining the level of p-CRMP2 present in the biological sample. In some embodiments, the method further comprises: computing a ratio of the p-CRMP2 to CRMP2 (p-CRMP2:CRMP2 ratio); and comparing the p-CRMP2:CRMP2 ratio to a reference p-CRMP2:CRMP2 ratio; wherein the reference p-CRMP2:CRMP2 ratio is derived from a second group of subjects known not to have the cognitive disorder.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological or psychological symptoms associated with the underlying disorder such that an improvement is observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing the cognitive disorder, or to a subject reporting one or more of the physiological or psychological symptoms of a disorder can undergo treatment, even though a diagnosis of this disorder was not confirmed.

In some aspects, treatment of the cognitive disorder such as SCZ involves a combination of medication or therapeutic agent, therapy, and self-management techniques. Two main types of medications or therapeutic agents that treat SCZ are i) typical antipsychotics or first generation antipsychotic drugs and ii) atypical antipsychotics or second generation antipsychotic drugs. Typical antipsychotics comprise haloperidol (Haldol), loxapine (Loxitane), thioridazine (Mellaril), molindone (Moban), thiothixene (Navane), fluphenazine (Prolixin), mesoridazine (Serentil), trifluoperazine (Stelazine), perphenazine (Trilafon), chlorpromazine (Thorazine), flupentixol, levomepromazine, periciazine, pimozide, prochlorperazine, promazine, sulpiride, and zuchl op enthixol. Atypical antipsychotics comprise pimavanserin (Nuplazid), ari pi prazol e (Abilify), clozapine (Clozaril), ziprasidone (Geodon), risperidone (Risperdal), quetiapine (Seroquel), olanzapine (Zyprexa), asenapine, iloperidone, amisulpride, lurasidone, and paliperidone.

In some aspects, therapeutic agents of the cognitive disorders such as SCZ can modulate glutamate, which is an abundant neurotransmitter in the brain. Increased dopamine release is known to be associated with symptoms of cognitive disorders such as SCZ. In another aspect, therapeutic agents of cognitive disorders such as SCZ can modulate dopamine release in the striatum. In some embodiments, therapeutic agents of cognitive disorders such as SCZ can reduce dopamine release in the striatum. In some embodiments, therapeutic agents of cognitive disorder such as SCZ can be used as an add-on treatment that reduces dopamine release in the striatum. In various aspects, therapeutic agents of cognitive disorders such as SCZ can modulate activity of muscarinic receptors.

In some embodiments, treatment of cognitive disorder such as SCZ comprises administration of therapeutic agents that modulate serotonin 5-HT_{2A} receptors such as Pimavanserin (inverse agonist and antagonist activity at serotonin 5-HT_{2A} receptors), therapeutic agents that modulate D-amino acid oxidase or DAAO activity such as Luvadaxistat (DAAO inhibitor), therapeutic agents that reduce dopamine released in the striatum such as Valbenazine, Tetrabenazine or Deutetrabenazine (vesicular monoamine transporter 2 or VMAT2 inhibitor), or therapeutic agents that modulate muscarinic receptors such as NBI-1117568 (muscarinic M4 agonist) to the human subject. In some embodiments, treatment of cognitive disorder such as SCZ comprises administration of Pimavanserin, Luvadaxistat, Valbenazine, Tetrabenazine, Deutetrabenazine or NBI-1117568. In some embodiments, the DAAO inhibitor is used for treatment of cognitive symptoms of SCZ. In some embodiments, the muscarinic M4 agonist is used for treatment of cognitive symptoms of SCZ. In some embodiments, the VMAT2 inhibitor is used for treatment of cognitive symptoms of SCZ. In some embodiments, the SCZ is treatment resistant SCZ.

As used herein, the term “administer”, “administered”, “administration”, or “to administer” refers to the step of giving (i.e. administering) a therapeutic agent or clinical investigational product (e.g. a pharmaceutical composition comprising a therapeutic agent or clinical investigational product) to a subject, or alternatively a subject receiving a therapeutic agent or clinical investigational product (e.g. a pharmaceutical composition comprising a therapeutic agent or clinical investigational product). The therapeutic agent or clinical investigational product (e.g. a pharmaceutical composition comprising a therapeutic agent or clinical investigational product) disclosed herein can be administered by various methods. For example, oral administration, intramuscular administration, intradermal administration, subcutaneous administration, intrathecal administration, intravenous administration, intraperitoneal administration, topical (transdermal), instillation, and implantation (for example, of a slow-release device such as polymeric implant or miniosmotic pump) can all be appropriate routes of administration.

In some embodiments, the human subject diagnosed with the cognitive disorders can be administered with one or more agents to reduce one or more symptoms of the cognitive disorders. In some embodiments, the agents are therapeutic agents or clinical investigational products. In some embodiments, the therapeutic agents or clinical investigational products are configured to mitigate or alleviated one or more symptoms of the cognitive disorder in the human subject. In some embodiments, the therapeutic agents can improve one of more symptoms of the cognitive disorder in the human subject.

In various embodiments, the cognitive disorder is not age-related. In some embodiments, the cognitive disorder is SCZ. In some embodiments, the SCZ is an early stage of SCZ. In some embodiments, the SCZ is treatment resistant SCZ. In some embodiments, the human subject diagnosed with SCZ can be administered with one or more agents to reduce one or more symptoms of SCZ. In some embodiments, the agents are therapeutic agents or clinical investigational products. In some embodiments, the therapeutic agents or clinical investigational products are configured to mitigate or alleviated one or more symptoms of SCZ in the human subject. In some embodiments, the therapeutic agents or clinical investigational products can improve one of more symptoms of SCZ in the human subject.

In some embodiments, the one or more agents are selected from the group consisting of typical antipsychotics and atypical antipsychotics. In some embodiments, the typical antipsychotics comprises haloperidol (Haldol), loxapine (Loxitane), thioridazine (Mellaril), molindone (Moban), thiothixene (Navane), fluphenazine (Prolixin), mesoridazine (Serentil), trifluoperazine (Stelazine), perphenazine (Trilafon), chlorpromazine (Thorazine), flupentixol, levomepromazine, periciazine, pimozide, prochlorperazine, promazine, sulpiride, and zuchlopenthixol. In some embodiments, the atypical antipsychotics comprises aripiprazole (Abilify), clozapine (Clozaril), ziprasidone (Geodon), risperidone (Risperdal), quetiapine (Seroquel), olanzapine (Zyprexa), asenapine, iloperidone, amisulpride, lurasidone, and paliperidone. In some embodiments, the one or more agents are selected from the group consisting of Pimavanserin, Luvadaxistat Valbenazine, Tetrabenazine, Deutetrabenazine and NBI-1117568.

In some aspects, therapeutic agents of the cognitive disorders such as SCZ can modulate glutamate, which is an abundant neurotransmitter in the brain. Increased dopamine release is known to be associated with symptoms of the cognitive disorders such as SCZ. In another aspect, therapeutic agents of the cognitive disorders such as SCZ can modulate dopamine release in the striatum. In some embodiments, therapeutic agents of cognitive disorders such as SCZ can reduce dopamine release in the striatum. In some embodiments, therapeutic agents of cognitive disorder such as SCZ can be used as an add-on treatment that reduces dopamine release in the striatum. In various aspects, therapeutic agents of the cognitive disorders such as SCZ can modulate activity of muscarinic receptors.

In some embodiments, treatment of the cognitive disorder such as SCZ comprises therapeutic agents that modulate D-amino acid oxidase or DAAO activity such as Luvadaxistat (DAAO inhibitor), therapeutic agents that reduce dopamine released in the striatum such as Valbenazine, Tetrabenazine or Deutetrabenazine (vesicular monoamine transporter 2 or VMAT2 inhibitor), or therapeutic agents that modulate muscarinic receptors such as NBI-1117568 (selective muscarinic M4 agonist). In some embodiments, the DAAO inhibitor is used for treatment of cognitive symptoms of the cognitive disorders such as SCZ. In some embodiments, the VMAT2 inhibitor is used for treatment of cognitive symptoms of the cognitive disorders such as SCZ. In some embodiments, treatment of the cognitive disorder such as SCZ comprises therapeutic agents that modulate serotonin 5-HT_{2A} such as Pimavanserin. In some embodiments, the SCZ is treatment resistant SCZ.

In various embodiments, the human subject diagnosed with the cognitive disorders can be administered one or more agents that affect the level of CRMP2. In some embodiments, the agents are configured to modulate the level of CRMP2. In some embodiments, the agents modulate the level of CRMP2. In some embodiments, the agents decrease the level of CRMP2. In some embodiments, the agents improve one or more symptoms of the cognitive disorders. In some embodiments, the agents are therapeutic agents. In various embodiments, the level of CRMP2 can be retested in the human subject diagnosed with the cognitive disorders, and drug regimen can be adjusted according to the level of CRMP2.

In some embodiments, the cognitive disorder is SCZ. In various embodiments, the human subject diagnosed with SCZ can be administered one or more agents that affect the level of CRMP2. In some embodiments, the agents are configured to modulate the level of CRMP2. In some embodiments, the agents modulate the level of CRMP2. In some embodiments, the agents decrease the level of CRMP2. In some embodiments, the agents improve one or more symptoms of SCZ. In some embodiments, the agents are therapeutic agents. In various embodiments, the level of CRMP2 can be retested in the human subject diagnosed with SCZ, and drug regimen can be adjusted according to the level of CRMP2.

In various embodiments, the human subject diagnosed with the cognitive disorders can be administered one or more agents that affect the ratio of p-CRMP2:CRMP2. In some embodiments, the agents are configured to modulate the ratio of p-CRMP2:CRMP2. In some embodiments, the agents modulate the ratio of p-CRMP2:CRMP2. In some embodiments, the agents increase the ratio of p-CRMP2:CRMP2. In some embodiments, the agents decrease the level of CRMP2. In some embodiments, the agents improve one or more symptoms of the cognitive disorders. In some embodiments, the agents are therapeutic agents. In various embodiments, the ratio of p-CRMP2:CRMP2 can be retested in the human subject diagnosed with the cognitive disorders, and drug regimen can be adjusted according to the ratio of p-CRMP2:CRMP2.

In some embodiments, the cognitive disorder is SCZ. In various embodiments, the human subject diagnosed with SCZ can be administered one or more agents that affect the ratio of p-CRMP2:CRMP2. In some embodiments, the agents are configured to modulate the ratio of p-CRMP2:CRMP2. In some embodiments, the agents modulate the ratio of p-CRMP2:CRMP2. In some embodiments, the agents increase the ratio of p-CRMP2:CRMP2. In some embodiments, the agents decrease the level of CRMP2. In some embodiments, the agents improve one or more symptoms of SCZ. In some embodiments, the agents are therapeutic agents. In various embodiments, the ratio of p-CRMP2:CRMP2 can be retested in the human subject diagnosed with SCZ, and drug regimen can be adjusted according to the ratio of p-CRMP2:CRMP2.

In some aspects, the present disclosure provides a method comprising administering a therapeutic agent that treats or slows the progression of one or more symptoms of a cognitive disorder to a human subject in need thereof. In some embodiments, a biological sample from the human subject in need thereof has been measured to detect the level of CRMP2 by an assay. In some embodiments, a biological sample from the human subject in need thereof has been measured to detect the levels of CRMP2 and phosphorylated CRMP2 (p-CRMP2) by the assay. In some embodiments, the ratio of p-CRMP2:CRMP2 is computed. In some embodiments, the ratio of p-CRMP2:CRMP2 of the human subject in need thereof is lower than 1.0. In some embodiments, the ratio of p-CRMP2:CRMP2 of the human subject in need thereof is lower than 0.9. In some embodiments, the ratio of p-CRMP2:CRMP2 of the human subject in need thereof is lower than 0.8. In some embodiments, the ratio of p-CRMP2:CRMP2 of the human subject in need thereof is lower than 0.7. In some embodiments, the ratio of p-CRMP2:CRMP2 of the human subject in need thereof is lower than 0.6. In some embodiments, the ratio of p-CRMP2:CRMP2 of the human subject in need thereof is lower than 0.5. In some embodiments, the ratio of p-CRMP2:CRMP2 of the human subject in need thereof is lower than 0.4. In some embodiments, the ratio of p-CRMP2:CRMP2 of the human subject in need thereof is lower than 0.3. In some embodiments, the ratio of p-CRMP2:CRMP2 of the human subject in need thereof is lower than 0.2. In some embodiments, the ratio of p-CRMP2:CRMP2 of the human subject in need thereof is lower than 0.1. In some embodiments, the ratio of p-CRMP2:CRMP2 of the human subject in need thereof is greater than 0.1.

In some embodiments, the cognitive disorder is not age-related. In some embodiments, the cognitive disorder is SCZ. In some embodiments, the SCZ is an early stage of SCZ. In some embodiments, the SCZ is treatment resistant SCZ.

In some embodiments, the human subject is less than 60 years old. In some embodiments, the human subject is less than 50 years old. In some embodiments, the human subject is less than 40 years old. In some embodiments, the human subject is less than 30 years old. In various embodiments, the human subject is about 17 years old to about 60 years old. In some embodiments, the human subject is about 17 years old to about 50 years old. In some embodiments, the human subject is about 17 years old to about 40 years old. In some embodiments, the human subject is about 17 years old to about 30 years old. In various embodiments, the human subject is about 18 years old to about 60 years old. In some embodiments, the human subject is about 18 years old to about 50 years old. In some embodiments, the human subject is about 18 years old to about 40 years old. In some embodiments, the human subject is about 18 years old to about 30 years old. In various embodiments, the human subject is at least 15 years old. In some embodiments, the human subject is at least 17 years old. In some embodiments, the human subject is at least 18 years old. In some embodiments, the human subject is at least 20 years old. In some embodiments, the human subject is at least 25 years old.

In various embodiments, the biological sample is a blood, saliva, urine, serum, tears, skin, tissue, or hair from the subject. In some embodiments, the biological sample is blood, saliva, or urine from the subject. In some embodiments, the biological sample is a blood sample from the subject comprising peripheral blood mononuclear cells (PBMC). In some embodiments, the PBMC comprises lymphocytes.

In some embodiments, the level of CRMP2 can be measured by the assay comprising a proteomic assay. In some embodiments, the proteomic assay is immunoassay, mass spectrometry, or intracellular flow cytometry. In some embodiments, the immunoassay comprises western blotting, dot blotting, quantitative enzyme-linked immunosorbent assays (ELISA), immunocytochemistry (ICC), immunohistochemistry (IHC), or protein multiplex assay, immunoprecipitation, radioimmunoassay (MA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression, or laser densitometry. In some embodiments, the assay is a lateral flow test.

In some embodiments, the levels of CRMP2 and p-CRMP2 can be measured by the assay comprising a proteomic assay. In some embodiments, the proteomic assay is immunoassay, mass spectrometry, or intracellular flow cytometry. In some embodiments, the immunoassay comprises western blotting, dot blotting, quantitative enzyme-linked immunosorbent assays (ELISA), immunocytochemistry (ICC), immunohistochemistry (IHC), or protein multiplex assay, immunoprecipitation, radioimmunoassay (MA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression, or laser densitometry. In some embodiments, the assay is a lateral flow test.

In some embodiments, the assay comprises an agent. In some embodiments, the agent is an antigen-binding agent. In some embodiments, the antigen-binding agent is an antibody. In some embodiments, the antigen-binding agent that recognizes CRMP2 is an anti-CRMP2 antibody and the antigen-binding agent that recognizes p-CRMP2 is an anti-p-CRMP2 antibody. In some embodiments, the antigen-binding agent that recognizes CRMP2 is an anti-CRMP2 monoclonal antibody and the antigen-binding agent that recognizes p-CRMP2 is an anti-p-CRMP2 monoclonal antibody. In some embodiments, the anti-p-CRMP2 antibody recognizes p-CRMP2 at Serine 522 (p-S522-CRMP2).

In some embodiments, the assay comprises a protein panel in which CRMP2 and p-CRMP2 are part of the protein panel. In some embodiments, CRMP2 can be one protein in a panel of protein that is detected. In some embodiments, p-CRMP2 can be one protein in a panel of protein that is detected. In some embodiments, both CRMP2 and p-CRMP2 can be proteins in a panel of proteins that are detected. In some embodiments, p-CRMP2 is phosphorylated at Serine 522 (p-S522-CRMP2).

In various embodiments, the human subject has one or more symptoms of the cognitive disorder comprises delusion, hallucination, disorganized thinking, disorganized speech, extremely disorganized or abnormal motor behavior, or social withdrawal. In some embodiments, the cognitive disorder is SCZ. In some embodiments, the method further comprises monitoring the level of CRMP2 from the human subject in need thereof over a period of at least 1 week, 1 month, 1 year, 5 years, or 10 years. In some embodiments, the method further comprises monitoring the levels of CRMP2 and p-CRMP2 from the human subject in need thereof over a period of at least 1 week, 1 month, 1 year, 5 years, or 10 years.

In some embodiments, the therapeutic agent is administered orally. In some embodiments, the therapeutic agent is administered as an intramuscular or submuscular injection. In some embodiments, the human subject is further received electroconvulsive therapy (ECT). In some embodiments, the human subject is further received complimentary interventions, e.g., vitamin D, vitamin E, vitamin B12 or B16, or folic acid supplements. In some embodiments, the human subject is further received non-pharmacological treatment, e.g., psychosocial interventions, individual therapy, meditation, yoga, cognitive behavior therapy, social skills training, family therapy, vocational rehabilitation, or supported employment.

### Kit

In another aspect, the present disclosure provides a kit comprising: a. one or more agents to collect a biological sample from a human subject; b. one or more agents to measure a level of CRMP2 and a level of p-CRMP2 from the biological sample obtained from the human subject; and c. an instruction to collect the biological sample and measure the levels of CRMP2 and p-CRMP2 using the agents in the kit. In some embodiments, the cognitive disorder is SCZ.

In some aspects, the present disclosure provides a kit for detecting level of CRMP2 from a biological sample obtained from a human subject with one or more symptoms of a cognitive disorder. In some embodiments, the kit comprises materials and reagents required for detecting level of CRMP2 from a biological sample obtained from a human subject with one or more symptoms of a cognitive disorder. In some embodiments, the cognitive disorder is SCZ.

In some aspects, the present disclosure provides a kit for detecting levels of CRMP2 and p-CRMP2 from a biological sample obtained from a human subject with one or more symptoms of a cognitive disorder. In some embodiments, the kit comprises materials and reagents required for detecting levels of CRM2 and p-CRMP2 from a biological sample obtained from a human subject with one or more symptoms of a cognitive disorder. In some embodiments, the cognitive disorder is SCZ. In some embodiments, the SCZ is treatment resistant SCZ.

The kit comprises agents, reagents, or materials to collect a biological sample from a human subject, and these agents can be solution and buffer that are known and used in the art. In some embodiments, the materials to collect the biological sample comprise sample collection containers, e.g, tube, cup, disposable vessel, bottle, traps, etc, that are known and used in the art. In some embodiments, the kit further comprises reagents that stabilize the biological sample such as blood during shipment. In some embodiments, these agents, reagents, or materials are used to process the biological sample for detecting level of CRMP2. In some embodiments, these agents, reagents, or materials are used to process the biological sample for detecting levels of CRMP2 and p-CRMP2. In some embodiments, the kit comprises one or more agents to measure the level CRMP2 from the biological sample obtained from the human subject. In some embodiments, the kit comprises one or more agents to measure the levels of p-CRMP2 and CRMP2 from the biological sample obtained from the human subject. These agents can be reagents, solutions, buffers, and materials that are known and used in the art for different types of assays that the kits are designed to perform, e.g., proteomic assay, immunoassay, mass spectrometry, intracellular flow cytometry, western blotting, dot blotting, quantitative enzyme-linked immunosorbent assays (ELISA), immunocytochemistry (ICC), immunohistochemistry (IHC), protein multiplex assay, or lateral flow tests.

In various embodiments, the kit comprises an instruction to collect the biological sample, to measure the level of CRMP2 using the agents, reagents, and materials in the kit, and to interpret the result from the assay for diagnosing the cognitive disorder.

In various embodiments, the kit comprises an instruction to collect the biological sample, to measure the levels of CRMP2 and p-CRMP2 using the agents, reagents, and materials in the kit, and to interpret the result from the assay for diagnosing the cognitive disorder. In some embodiments, the present disclosure provides a kit comprising i) one or more agents to collect a biological sample from a human subject; ii) one or more agents to measure a level of CRMP2 and a level of p-CRMP2 from the biological sample obtained from the human subject; and iii) an instruction to collect the biological sample and measure the levels of CRMP2 and p-CRMP2 using the agents in the kit. In some embodiments, the cognitive disorder is SCZ. In some embodiments, the SCZ is treatment resistant SCZ.

In some embodiments, the kit contains another instruction for interpreting the result and for diagnosing the cognitive disorder. In some embodiments, the kit contains another instruction for interpreting the result and for diagnosing SCZ. In specific embodiments, the kit can further comprise one or more additional reagents employed in various methods, such as (1) reagents to purify protein and posttranslational modification of protein from the biological sample; (2) reagents to purify DNA or mRNA to test for additional biomarkers; (3) labeled probe purification reagents and components, e.g., spin columns; (4) signal generation and detection reagents, e.g., streptavidin-alkaline phosphatase conjugate, chemifluorescent or chemiluminescent substrate, and the like; and (5) reagents and materials for lateral flow test or test strip, e.g., sample pad, conjugated pad, nitrocellulose membrane, absorbent pad, colored nanoparticles/labels, and antibodies. In some embodiments, the kit comprises prelabeled quality controlled protein and phosphorylated protein isolated from a sample for use as a control.

As used herein, the terms “lateral flow test” or “test strip” refers to a diagnostic test used to confirm presence of absence of a target analyte, which can be pathogens, proteins, antigens, fragments of protein or antigen, or biomarkers in the biological sample obtained from the subject. Other names referred to lateral flow test are lateral flow device, lateral flow assay, lateral flow immunoassay, lateral flow immunochromatographic assay, dipstick, express test, pen-side test, quick test, test strip, and rapid test. In some embodiments, the immunoassay can be a lateral flow test. In some embodiments, the kit is a lateral flow test or a test strip. Exemplary embodiments include but not limited to the lateral flow test described in for example, U.S. Pat. No. 7,955,791 B2 and U.S. Pat. No. 10,670,596 B2, which are incorporated by reference in its entirety.

In some embodiments, the kit can comprise an array having a protein panel, and CRMP2 and p-CRMP2 are parts of the protein panel in the array. In some embodiments, the kit can be a multiplex assay detecting one or more additional biomarkers as described in, for example, U.S. Pat. No. 8,258,284 B2, which is incorporated by reference in its entirety. In some embodiments, the kit can be used for monitoring the level of CRMP2 for over certain periods, e.g., 1 month, 1 year, 5 years, or 10 years. In some embodiments, the kit can be used for monitoring the levels of CRMP2 and p-CRMP2 for over certain periods, e.g., 1 month, 1 year, 5 years, or 10 years. In some embodiments, the kit can be used for monitoring the efficacy of one or more therapeutic agents that the human subject is undergoing based upon the levels of CRMP2. In some embodiments, the kit can be used for monitoring the efficacy of one or more therapeutic agents that the human subject is undergoing based upon the levels of p-CRMP2 and CRMP2 or ratio of p-CRMP2:CRMP2. In some embodiments, the kit can be used for determining whether the human subject will be responsive to one or more therapeutic agents based upon the level of CRMP2 and/or ratio of p-CRMP2:CRMP2.

In some embodiments, the kit comprises an agent. In some embodiments, the agent is an antigen-binding agent. In some embodiments, the antigen-binding agent is an antibody. In some embodiments, the antigen-binding agent that recognizes CRMP2 is an anti-CRMP2 antibody and the antigen-binding agent that recognizes p-CRMP2 is an anti-p-CRMP2 antibody. In some embodiments, the antigen-binding agent that recognizes CRMP2 is an anti-CRMP2 monoclonal antibody and the antigen-binding agent that recognizes p-CRMP2 is an anti-p-CRMP2 monoclonal antibody. In some embodiments, the anti-p-CRMP2 antibody recognizes p-CRMP2 at Serine 522 (p-S522-CRMP2).

In various embodiments, the agents to measure the levels of CRMP2 is antigen-binding agent. In some embodiments, the antigen-binding agent to measure the level of CRMP2 is an antibody. In some embodiments, the antigen-binding agent that recognizes CMRP2 is an anti-CRMP2 antibody. In some embodiments, the antigen-binding agent that recognizes CRMP2 is an anti-CRMP2 monoclonal antibody. In some embodiments, the agents to measure the levels of CRMP2 and p-CRMP2 are antigen-binding agents. In some embodiments, the antigen-binding agents to measure the levels of CRMP2 and p-CRMP2 are antibodies. In some embodiments, the antigen-binding agent that recognizes CRMP2 is an anti-CRMP2 antibody and the antigen-binding agent that recognizes p-CRMP2 is an anti-p-CRMP2 antibody. In some embodiments, the antigen-binding agent that recognizes CRMP2 is an anti-CRMP2 monoclonal antibody and the antigen-binding agent that recognizes p-CRMP2 is an anti-p-CRMP2 monoclonal antibody. In some embodiments, the anti-p-CRMP2 antibody recognizes p-CRMP2 at Serine 522 (p-S522-CRMP2). In some embodiments, the antibodies comprise an anti-CRMP2 and an anti-p-CRMP2. In some embodiments, the anti-p-CRMP2 is an anti-p-Serine 522-CRMP2 (p-S522-CRMP2).

In some embodiments, the biological sample can be obtained from the human subject to use for the kit described herein. In some embodiments, the biological sample obtained from the human subject can be blood, saliva, urine, serum, tears, skin, tissue, or hair. In some embodiments, the biological sample obtained from the human subject is blood, saliva, or urine. In some embodiments, the blood sample comprises peripheral blood mononuclear cells (PBMC). In some embodiments, the PBMC comprises lymphocytes. In some embodiments, the biological sample obtained from the human subject can comprise cells or tissues that express CRMP2. In various embodiments, one or more agents to collect the biological sample are designed to be suitable to process each type of the sample accordingly and these agents are used and known in the art.

In some embodiments, the human subject is a male or female. In some embodiments, the human is adolescence. In some embodiments, the human is adult. In some embodiments, the human subject is less than 60 years old. In some embodiments, the human subject is less than 50 years old. In some embodiments, the human subject is less than 40 years old. In some embodiments, the human subject is less than 30 years old. In various embodiments, the human subject is about 17 years old to about 60 years old. In some embodiments, the human subject is about 17 years old to about 50 years old. In some embodiments, the human subject is about 17 years old to about 40 years old. In some embodiments, the human subject is about 17 years old to about 30 years old. In various embodiments, the human subject is about 18 years old to about 60 years old. In some embodiments, the human subject is about 18 years old to about 50 years old. In some embodiments, the human subject is about 18 years old to about 40 years old. In some embodiments, the human subject is about 18 years old to about 30 years old. In various embodiments, the human subject is at least 15 years old. In some embodiments, the human subject is at least 17 years old. In some embodiments, the human subject is at least 18 years old. In some embodiments, the human subject is at least 20 years old. In some embodiments, the human subject is at least 25 years old.

In some embodiments, the kit is part of a health-related kit that can be performed by a healthcare provider. In some embodiments, the kit can be performed at home by patient. In some embodiments, the kit can be performed at home by a caregiver.

### EXAMPLES

The following examples are included for illustrative purposes only and are not intended to limit the scope of the invention.

### Example 1: Brain Samples from SCZ Patients Showed Dendritic Abnormalities

In some cases, the brains of patients with SCZ show dendritic abnormalities and differences in CRMP2 expression when compared to the brains of unaffected patients without dendritic abnormalities. To study the brain samples from SCZ patients, frozen, postmortem, human brain tissues obtained from the Harvard Brain Tissue Resource Center were blindly examined.

Archived Clinical Patient Brain Tissue Processing

For Cohort 1, frozen, postmortem, human brain tissue containing the dorsolateral prefrontal cortex or DLPFC (Brodman Area 46) was obtained from the Harvard Brain Tissue Resource Center. Because these patients cannot be identified, no IRB approval was required. Permission to use the samples for research was given by the donors at the time they made their anatomical gifts. The demographics of the patients whose donated brains were used is summarized in Table 1.

TABLE 1
Demographic information on patients
whose postmortem brains were analyzed
Demographic information on patients
whose brains were analyzed

| | Schizophrenia | Control | |
|---|---|---|---|
| Sex (M/F) | 13/6 | 14/5 | p-value |
| Age | 59.5 ± 12.7 | 55.8 ± 12.3 | n.s. |
| Postmortem interval (hours) | 23.9 ± 6.6 | 22.5 ± 3.9 | n.s. |
| Storage time (months) | 137.3 ± 30.5 | 119.7 ± 18.6 | n.s. |
| pH | 6.5 ± 0.3 | 6.4 ± 0.2 | n.s. |

### Tissue Processing

Tissue samples were dissected to produce two tissue blocks measuring 1.5 cm²×0.5 cm and 1.5 cm²×0.2-0.3 cm, respectively. The first block was stained using the Golgi-Kopsch method. Tissue blocks were shaken in a 4% potassium dichromate in 5% paraformaldehyde solution at room temperature in the dark for 96 hours. The potassium dichromate/paraformaldehyde solution was replaced every 36 hours and acid-cleaned glassware was utilized. Tissue blocks were washed in increasing concentrations of silver nitrate (0.25, 0.5, 0.75 and 1%) and then shaken in 1% silver nitrate in acid-cleaned glassware at room temperature in the dark for 1 week. Stained tissue blocks were sectioned using a vibrating microtome (Vibratome, St. Louis, MO) at 100 μm, mounted on gelatin-coated slides, and briefly air-dried. Tissue sections were then dehydrated with a graded series of ethanol, cleared with xylene and cover-slipped with Permount (Fisher Scientific). The second block was sectioned using a vibrating microtome at 40 mounted on gelatin-coated slides, and air-dried. The tissue sections were then stained for cytoplasmic ribonucleic acid (Nissl substance) using thionin and cover-slipped. The dorsolateral prefrontal cortex or DLPFC area of the brain sample was studied.

Pyramidal Cell Reconstruction

Archived frozen postmortem human brain tissue containing the dorsolateral prefrontal cortex (DLPFC) (obtained from the Harvard Brain Tissue Resource Center) was processed to enable pyramidal cell reconstruction. Nissl-stained sections were used to confirm localization to DLPFC using established cytoarchitectonic criteria and to ascertain the borders of layer III as a percentage of cortical thickness. Fifteen Golgi-stained pyramidal cells were selected for reconstruction per subject using the following criteria: (a) somata located in the bottom half of layer III and in the middle of the section thickness; (b) pyramidal cell fully impregnated; (c) somata or dendrites not obscured by large (>5 μm) staining opacities; (d) no morphological changes associated with postmortem interval; and (e) presence of ≥3 basilar dendrites each branching at least once. For each selected pyramidal cell, the apparently longest basilar dendrite was selected visually and reconstructed using Neurolucida (version 11, MicroBrightfield Bioscience, Williston, VT) with a Zeiss Axioskop 2 Plus light microscope (Carl Zeiss, Germany) and a ×100 oil immersion objective (NA=1.4, working distance=0.17 mm). Reconstructions were done on live images captured using an CX9000 digital camera (MicroBrightfield Bioscience, Williston, VT) at a final resolution of 1600×1200. Each dendrite terminus was classified as ending naturally or artificially at the cut surface of the section.

As shown in FIGS. 1A-1B, brain biopsy from SCZ patient shows dendritic abnormalities (FIG. 1B) compared to the brain biopsy from unaffected age-match controls (FIG. 1A), with arrows point to dendritic spines. As shown in FIG. 1B, the dendritic spines are markedly reduced in the SCZ patients.

The results in FIGS. 1A-1B from staining of the samples showed that both shortened basilar dendrites and basilar dendritic spine loss were detected on pyramidal neurons (with somata in the deep layer III of the DLPFC) from SCZ patients.

### Example 2: Relationship of CRMP2 and p-CRMP2 Protein Level from the Brain Sample and Dendritic Parameters

To determine whether the difference in CRMP2 expression might be associated with this dendritic arbor pathology in SCZ, quantitative western blotting was performed. A correlation analysis was also calculated to determine the correlation of protein expression from the same tissue with dendritic parameters.

Archived Clinical Patient Brain Tissue Processing and Protein Extraction

For Cohort 1, the brain samples were obtained as previously described from the previous example. For total protein extraction, Halt protease and phosphatase inhibitor cocktails (Fisher) were added and total protein concentration was quantified. Samples were separated on 4-20% Mini-Protein TGX gels (Bio-Rad), then transferred to PVDF membranes. Then membranes were blocked and probed with primary antibodies. After incubation with HRP-conjugated secondary antibodies, immunocomplexes were visualized using Western Lightning Plus ECL (Perkin Elmer). Semi-quantitative assessment of protein bands was executed by computerized densitometry using the ChemiDoc XRS+ System (Bio-Rad) and Image Lab Software (Bio-Rad). Chemiluminescent values of the protein bands were divided by its corresponding NSE chemiluminescent values. The SCZ values were normalized to control values (% control) collected in parallel from the same gel.

Antibodies

An anti-human CRMP2 monoclonal antibody (9F) was raised by injection of a C-terminal region of human CRMP2 (amino acid 486-528) into Balb/c mice (18). An anti-phosphorylated CRMP1/2(S522) (p-S522-CRMP1/2) polyclonal antibody that recognizes CRMP2 phosphorylated at Ser522 was generated in rabbits. The specificities of the antibodies were verified by peptide-blocking experiment in immunoblot analysis of human PBMC samples (See FIGS. 2A-2B and FIGS. 3A-3B). Immunoblots of wild-type and CRMP2-KO mouse brains were used to confirm the specificity of the anti-CRMP2 antibodies. The use of these animals for this purpose was approved by the institutional Animal Care and Use Committee (IACUC) of the Yokohama City University Graduate School of Medicine.

Immunoblot Analysis

Sample pellets were homogenized in 50 μL immunoprecipitation IP buffer (20 mM Tris-HCl, pH 8.0, 150 mM NaCl, 1 mM EDTA, 10 mM NaF, 1 mM Na₃VO₄, 1% Nonidet P-40, 50 μM ρ-APMSF (ρ-amidinophenylmethanesulfonyl fluoride)), and were centrifuged at 15000 g for 20 min at 4° C. The samples were then used for immunoblot analysis with anti-CRMP2 (9F), anti-phosphorylated CRMP1/2, and anti-O-actin mouse monoclonal (A5316, Sigma-Aldrich, MO, USA) antibodies with signal enhancer HIKARI (Nacalai Tesque, Kyoto, Japan). Actin (2.0 mg/ml) and rat brain lysate (0.5 mg/mL) were used as loading controls. The relative amounts of CRMP2 and its Ser522 phosphorylated form of CRMP2 (p-S522-CRMP2) were estimated using beta-actin (β-actin) as an internal standard. (See FIGS. 2A-2B and 3A-3B)

Correlation with Dendrite Parameters

For each differentially-expressed protein or ratio, the relationship between its value and basilar dendrite parameters for pyramidal cells in the deep half of layer III in the DLPFC was assessed. A Pearson correlation coefficient was calculated for the relative protein expression level or ratio and the number of spines per basilar dendrite, basilar spine density, and basilar dendrite length

Statistical Analyses of Data from Clinical Human Postmortem Brain Tissue

All statistical analyses for data from human patient postmortem brain tissue were conducted using STATA (v. 12, College Station, TX). As stated above, normalized relative protein expression values were used. Intergroup protein expression differences were assessed using an ANCOVA model. Covariates were selected from age, sex, postmortem interval (PMI), storage time, and pH using Aikake's Information Criterion corrected for small samples (AICc).

Results from quantitative western blotting as seen in FIGS. 4, 5A-5B showed the relative protein expressions of CRMP2 and p-CRMP2, and the resultant p-CRMP2:CRMP2 ratio were assessed in the above-mentioned archived DLPFC grey matter specimens from patients with SCZ (n=19) and from unaffected control subjects (n=19) (Note: there were no demographic or pharmacological differences between the two cohorts [See Table 1]).

Results from correlation analysis of protein expression with dendrite parameters showed that protein expression and ratios were then correlated with dendritic parameters (e.g., basilar dendrite spine density, dendrite length, and the number of spines-per-dendrite) of pyramidal neurons in the deep layer III of the DLPFC. Relative to unaffected controls, CRMP2 from SCZ patient was significantly increased by 10% (p=0.05) as seen in FIG. 4. In addition, CRMP2 protein expression from SCZ patient was inversely correlated with basilar dendrite length (r=−0.37, p=0.04) as seen in FIG. 1B.

Although CRMP2 levels were significantly higher in brains from patients with SCZ compared to brains from control subjects, phosphorylated CRMP2 (p-CRMP2) protein expression as seen in FIG. 5A and, hence, the ratio of p-CRMP2:CRMP2 protein expression as seen in FIG. 5B was not statistically different in brains from SCZ patients compared to brains from control subjects (*p>0.05), when the data were analyzed in the aggregate.

### Example 3: Impact of the Age of Cohort on CRMP2 and p-CRMP2 Levels

There was a difference between the brains of patient with SCZ compared to bipolar disorder (BPD). In the DPLFCs of patients with lithium responsive bipolar disorder (LiR BPD), the CRMP2 was not significantly different from age-matched unaffected patients but p-CRMP2 was increased. Hence, the p-CRMP2:CRMP2 ratio was significantly elevated in LiR BPD patients compared to unaffected controls (as well as compared to patients with other psychiatric and neurological diseases, including lithium non-responsive [LiNR] BPD) because the numerator—p-CRMP2—was abnormally high while the denominator—CRMP2—remained unchanged. The results showed the ostensible lack of a statistically significant decrease in the p-CRMP2:CRMP2 ratio between SCZ brains and age-matched control brains despite the fact that CRMP2, the denominator, was increased. This finding suggested the following two possibilities: (i) The brain's machinery for phosphorylating (and, hence, inactivating) the increased CRMP2 substrate seen in SCZ could keep pace with the greater burden, or (ii) p-CRMP2 was also rising for reasons independent of SCZ. Apropos to that latter possibility, the mean age of this cohort was relatively old (59.5±12.7 years-of-age) [See Table 1] and p-CRMP2 often rises in association with aging-related conditions.

Furthermore, the nature of psychiatric brain banking and the stochastic, often serendipitous, manner in which specimens are voluntarily donated as anatomical gifts makes most such SCZ collections skew to older brains. (The BPD study comprised younger patients because that disorder's demographics skew younger with many sufferers dying prematurely from suicide or substance abuse, less so seen in SCZ). Notwithstanding the limitations in SCZ brain banking, there was an observation within this retrospective unselected cohort suggesting to focus the study on the few “young” patients (defined in psychiatry as <40 years old) in Cohort 1 [FIGS. 4, 5A, and 5B]: one with SCZ (the arrow and square) and one unaffected (the arrow and dot)—results showed that while CRMP2 (the denominator) was significantly elevated in the young SCZ patient (as with all of the 19 SCZ patients), p-CRMP2 (the numerator) was indistinguishable from the young unaffected subject, making the p-CRMP2:CRMP2 ratio much lower than normal (0.8 compared to 1.3) in the young SCZ patient.

### Example 4: CRMP2 Protein Level from the Peripheral Blood (PBMC) Sample in Young Patients

Cohorts for SCZ could be stratified by age to avoid the results being potentially confounded by conditions or morbidities unrelated to SCZ.

CRMP2 is expressed in lymphocytes. Therefore, the elevated CRMP2 levels detected in the brains of patients with SCZ could be reflected in the PBMCs of such patients. Thus, the immunoblotting and analysis were performed from the PBMC samples obtained from another cohort (Cohort 2) stratified by age.

Patients from Whom PBMCs were Collected

For Cohort 2, the demographics of the SCZ and healthy control patients (ranging in age from 18-40 years old) from whom PBMCs were collected are summarized in Table 2. Participants in cohort 2 were “young” (defined as <40 years old). “Middle-aged” (40-60 years old) and “elderly” (>60 years old) patients were not included in Cohort 2. Such data includes age and sex, family medical history, smoking history, drinking history, education, career, medication history. They were inpatients or outpatients at Yokohama City University Hospital, Yokohama Medical Center, or Yokohama Maioka Hospital (affiliated with Yokohama City University). The study was approved by the Ethical Review Board of each hospital. Each SCZ patient was diagnosed by at least two psychiatrists based on the diagnostic criteria of Diagnostic & Statistical Manual of Mental Disorders (DSM-IV). The PANSS (Positive and Negative Symptom Scale) was used to assess symptom severity. For matched unaffected controls, volunteers were enlisted by a general open call. Samples and data from age- and sex-matched normal (as determined by examination from 2 blinded psychiatrists) volunteers were similarly obtained (as sanctioned). Each volunteer was interviewed by psychiatrists to exclude mental disorders. Informed consent was obtained from all participants. Samples were assessed blindly by the investigators.

TABLE 2
Demographic information on patients whose PBMCs were analyzed

| | Control | Control | Control |
|---|---|---|---|
| | Subjects | Subjects | Subjects |
| | (Total) | (≤30 years old) | (30<, ≤40) |
| N | 37 | 25 | 12 |
| Sex | M: 19 | M: 12 | M: 7 |
| | F: 18 | F: 13 | F: 5 |
| Age (years) | 29.1 | 25.5 | 36.7 |
| Education (years) | 19.3 | 17.6 | 20.3 |

| | Schizophrenia | Schizophrenia | Schizophrenia |
|---|---|---|---|
| | Patients | Patients | Patients |
| | (Total) | (≤30 years old) | (30<, ≤40) |
| N | 21 | 13 | 8 |
| Sex | M: 9 | M: 4 | M : 5 |
| | F: 12 | F: 9 | F: 3 |
| Age (years) | 28.5 | 23.7 | 36.4 |
| Disease period (years) | 8.4 | 4.0 | 15.6 |
| Quantity of medication | 668.3 | 834.2 | 398.6 |
| (CP conversion: mg) | | | |
| GAF^{※1} | 44.7 | 40.5 | 52.4 |
| PANSS^{※2} | | | |
| Positive subscale score | 19.4 | 20.2 | 16.8 |
| Negative subscale score | 21.6 | 22.7 | 18.0 |
| General psychopathology score | 44.2 | 46.1 | 37.8 |
| Total score | 84.6 | 89.0 | 70.0 |
| Education (years) | 14.1 | 13.9 | 14.4 |

^{※1}GAF: The Global Assessment of Functioning
^{※2}PANSS: Positive and Negative Syndrome Scale

Blood Sample Collection and Lymphocyte Separation

PBMCs were isolated from all collected blood samples using a mononuclear cell preparation tube (BD Vacutainer CPT:Becton—Dickinson, NJ, USA). The whole blood specimens were centrifuged for 30 min (1500 g, 25° C.) to collect a PBMC layer in 15-mL tubes. These samples were then washed with phosphate buffered saline minus (PBS) and centrifuged three times at 427 g for 10 min at 4° C. After discarding the supernatant, the pellet was resuspended in RPMI1640 medium supplemented with 10% fetal bovine serum (FBS) and ampicillin. The resuspended samples were plated on the non-coated Petri dish and left in CO2 incubator at 37° C. for two days. The supernatant fraction containing non-adherent lymphocytes was collected and transferred to 15-mL tubes. The samples were centrifuged at 200 g for 5 min at 4° C. After discarding the supernatants, the pellets were resuspended in 1 mL PBS, and transferred to 1.5 mL tubes. After centrifuging at 200 g for 5 min at 4° C., the supernatants were discarded and the pellets were frozen with liquid nitrogen and stored at −80° C. until use. CRMP2 and p-CRMP2 levels were subsequently assessed.

Antibodies

Antibodies from the previous examples were used. The specificities of the antibodies were verified by peptide-blocking experiment in immunoblot analysis of human PMBC samples (See FIGS. 2A-2B and 3A-3B).

Immunoblot Analysis

Immunoblot experiments were performed as described in the previous examples. Purified CRMP2 was used as a standard to calculate concentration of CRMP2 in lymphocyte lysate in immunoblotting by using ImageJ.

Statistical Analysis of Data from Peripheral Blood Specimens

Statistical analysis was conducted using IBM SPSS Statistics 22 (IMB Analysis). For a Mann-Whitney U test, statistical significance was considered to be p<0.05. For examination between continuous variables, a regression analysis was conducted; significance of the correlation coefficient was considered to be p<0.05.

Results from FIGS. 6A-6B of immunoblotting of PBMC showed, interestingly, the differences in CRMP2 between patients with SCZ and unaffected age-matched controls, as detected through assaying this more accessible peripheral blood-derived cell type, was actually more striking than in the brain; in addition, this approach in this cohort revealed the p-CRMP2:CRMP2 ratio to be the informative metric.

FIGS. 6A-6B showed CRMP2 and p-CRMP2 in peripheral blood mononuclear cell (PBMC) samples from living patients. In order to examine patients that likely had “pure” SCZ, and also to eliminate the confounding effects of aging-related diseases as well as poly-pharmacy (including for non-psychiatric conditions), evaluation on patients with SCZ <40 years of age was the focus, which is a time before diseases-of-aging and unrelated co-morbidities could intervene. Demographic information for this group of SCZ patients and for their unaffected age-matched controls is summarized in Table 2 (total n=58). There were no significant differences between the two groups in that regard.

Significant differences did exist between SCZ patients (n=21) and unaffected controls (n=37) with regard not only to the levels of CRMP2 but also the resultant p-CRMP2: CRMP2 ratios as seen in FIGS. 6A-6B. As in the brains, CRMP2 levels were significantly higher in the PBMCs of SCZ patients—most strikingly so in SCZ patients <30 years old (*p<0.01). On the other hand, phosphorylation of CRMP2 did not increase concomitantly with an increase in the CRMP2 substrate, and the ratio of p-CRMP2: CRMP2 was, therefore, lower in the SCZ group (compared to the unaffected age-matched control group) because the denominator (CRMP2) was larger based on its increased abundance as seen in FIGS. 6A-6B. With an excess of active (non-phosphorylated) CRMP2— in contrast to the equilibrium between active and inactive (phosphorylated) CRMP2 that normally exists—neural network function, particularly at dendritic spines, might be aberrant. Notably, the lower p-CRMP2:CRMP2 ratio in SCZ contrasted dramatically with the higher ratio in patients with LiR BPD, enhancing the potential utility of this diagnostic aid.

In short, in blood, excessive amounts of active (non-phosphorylated) CRMP2 distinguishes SCZ patients (with suitable sensitivity and specificity) from unaffected age-matched subjects, and likely from psychiatric disorders that might mimic SCZ in a newly-presenting young patient, for example, BPD.

Overall, as shown in FIG. 4, among the SCZ patients, the brain indicated by the arrow and square came from a 23 year-old female with a CRMP2 level elevated to 129.7 (162% of control, as per FIG. 4) but with a p-CRMP2 level within normal limits (104.5, which is 97.2% of control) per FIG. 5A. Since p-CRMP2 often rises in association with aging-related conditions, the age of the human subject is a confounding factor and can mask the effect. As shown in FIGS. 6A-6B, by eliminating this factor and comparing the p-CRMP2:CRMP2 ratio from SCZ patient to the unaffected age-control group, the p-CRMP2:CRMP2 ratio in lymphocytes from patients with SCZ is lower than the ratio of p-CRMP2:CRMP2 in lymphocyte from the unaffected age-control group. Limiting the age of the human subject to below 40 years of age provides correct assessment of p-CRMP2:CRMP2 ratio.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A method for determining an increased risk for a cognitive disorder in a human subject, comprising:
collecting a biological sample from the human subject, wherein the biological sample is a blood, saliva, urine, serum, tears, skin, tissue, or hair from the human subject;
determining, by an assay, a level of collapsing response mediator protein-2 (CRMP2) in the biological sample, wherein the assay is a proteomic assay and comprises:
i. contacting the biological sample with an agent that recognizes CRMP2, and
ii. measuring the level of bound CRMP2 and thereby determining the level of CRMP2 present in the biological sample, wherein the agent that recognizes CRMP2 is an antigen-binding agent; and
detecting the level of CRMP2.

2. The method as in claim 1, where in the assay further comprises:
i. contacting the biological sample with an agent that recognizes phosphorylated CRMP2 (p-CRMP2),
ii. measuring a level of bound p-CRMP2 and thereby determining the level of p-CRMP2 present in the biological sample; and
iii. detecting the level of p-CRMP2.

3. The method as in claim 2, further comprising:
a. computing a ratio of the p-CRMP2 to CRMP2 (p-CRMP2:CRMP2 ratio); and
b. detecting a reduced p-CRMP2:CRMP2 ratio in comparison to a reference p-CRMP2:CRMP2 ratio.

4. (canceled)

5. The method as in claim 1, wherein the cognitive disorder is Schizophrenia (SCZ).

6. The method as in claim 5, wherein the SCZ is an early stage of SCZ.

7. (canceled)

8. The method as in claim 1, wherein the biological sample is a blood sample from the human subject comprising peripheral blood mononuclear cells (PBMC).

9. (canceled)

10. (canceled)

11. (canceled)

12. (canceled)

13. (canceled)

14. (canceled)

15. (canceled)

16. (canceled)

17. The method as in claim 1, wherein the proteomic assay is immunoassay, mass spectrometry, or intracellular flow cytometry.

18. The method as in claim 17, wherein the immunoassay is selected from western blotting, dot blotting, quantitative enzyme-linked immunosorbent assays (ELISA), immunocytochemistry (ICC), immunohistochemistry (IHC), protein multiplex assay, or lateral flow test.

19. (canceled)

20. The method as in claim 2, wherein the antigen-binding agent that recognizes CRMP2 is an anti-CRMP2 antibody; and the antigen-binding agent that recognizes p-CRMP2 is an anti-p-CRMP2 antibody.

21. The method as in claim 20, wherein the antigen-binding agent that recognizes CRMP2 is an anti-CRMP2 monoclonal antibody; and the antigen-binding agent that recognizes p-CRMP2 is an anti-p-CRMP2 monoclonal antibody.

22. The method as in claim 3, further comprising identifying the human subject with added risk of having the cognitive disorder if the p-CRMP2:CRMP2 ratio of the subject is decreased compared to the reference p-CRMP2:CRMP2 ratio.

23. The method as in claim 22, further comprising identifying the human subject with added risk of having the cognitive disorder if the p-CRMP2:CRMP2 ratio of the subject is lower than 1.0, 0.9, 0.8, 0.7, 0.6, 0.5, 0.4, 0.3, 0.2, 0.1 or within a range defined by any of the preceding values, or further comprising identifying the human subject with added risk of having the cognitive disorder if the p-CRMP2:CRMP2 ratio of the subject is about 40% lower than the reference p-CRMP2:CRMP2 ratio.

24. The method as in claim 22, further comprising identifying the human subject with added risk of having the cognitive disorder if the p-CRMP2:CRMP2 ratio of the subject is between 0.1 to 0.4, or 0.2 to 0.5.

25. (canceled)

26. (canceled)

27. The method as in claim 2, wherein the CRMP2 is phosphorylated at Serine 522 (p-S522-CRMP2), wherein measuring the level of p-CRMP2 comprises measuring a phosphorylation of CRMP2 at Serine 522.

28. (canceled)

29. (canceled)

30. (canceled)

31. (canceled)

32. (canceled)

33. (canceled)

34. (canceled)

35. (canceled)

36. (canceled)

37. (canceled)

38. The method as in claim 1, further comprising treating the human subject with one or more antipsychotic drugs.

39. (canceled)

40. (canceled)

41. (canceled)

42. (canceled)

43. (canceled)

44. (canceled)

45. (canceled)

46. A method comprising:
collecting a biological sample from a human subject;
determining, by an assay, a level of CRMP2 in the biological sample from the subject, wherein the assay comprises:
i. contacting the biological sample with an agent that recognizes CRMP2,
ii. measuring the level of bound CRMP2 and thereby determining the level of CRMP2 present in the biological sample, and
iii. computing the risk of the human subject having a cognitive disorder based on determining the level of CRMP2 in comparison to a reference level of CRMP2; and
administering a therapeutic agent to the human subject,
wherein the therapeutic agent is configured to mitigate or alleviate one or more symptoms of the cognitive disorder in the human subject.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. A method of treating a cognitive disorder in a human subject, comprising:
performing the method as in claim 1; and
administering a therapeutic agent or clinical investigational product to the human subject.

79. (canceled)

80. (canceled)

81. The method as in claim 78, wherein the therapeutic agent or clinical investigational product is a D-amino acid oxidase (DAAO) inhibitor, vesicular monoamine transporter 2 (VMAT2) inhibitor, or muscarinic M4 agonist.

82. The method as in claim 81, wherein the DAAO inhibitor is Luvadaxistat, the VMAT2 inhibitor is Valbenazine, Tetrabenazine or Deutetrabenazine, and the muscarinic M4 agonist is NBI-1117568.

83. (canceled)

84. (canceled)

85. A kit comprising:
a. one or more agents to collect a biological sample from a human subject;
b. one or more agents to measure a level of CRMP2 and a level of p-CRMP2 from the biological sample obtained from the subject; and
c. an instruction to collect the biological sample and measure the levels of CRMP2 and p-CRMP2 using the agents in the kit.

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)
